(19) United States
(12) Patent Application Publication
(10) Pub. No.: US 2003/0119061 A1
(43) Pub. Date: Jun. 26, 2003

(54) Structure-based drug design methods for identifying D-Ala-D-Ala ligase inhibitors as antibacterial drugs

(76) Inventors: Paul J. Ala, Newark, DE (US); Janid A. Ali, Waltham, MA (US); Carlos H. Faerman, Acton, MA (US); James P. Griffith, Weston, MA (US); Andrew S. Magee, Maynard, MA (US); Scott T. Moe, Marlborough, MA (US); Manuel A. Navia, Lexington, MA (US); Emanuele Perola, Cambridge, MA (US); Patrick R. Connelly, Harvard, MA (US)

Correspondence Address: FISH & RICHARDSON PC, 225 FRANKLIN ST, BOSTON, MA, 02110, US

(21) Appl. No.: 10/186,886

(22) Filed: Jun. 28, 2002

Related U.S. Application Data
(60) Provisional application No. 60/301,676, filed on Jun. 28, 2001.

(51) Int. Cl. G01N 33/53; G06F 19/00; G01N 33/48; G01N 33/50

(52) U.S. Cl. 435/7.1; 702/19

(57) ABSTRACT

The invention is based on the discovery that certain small molecules can bind to the ATP binding site of D-Ala-D-Ala ligase, even in the absence of the enzyme's substrate, and can cause a conformational change in the enzyme structure similar to that which occurs upon binding of ATP and substrate to the enzyme. Without wishing to be bound by any theory, it is believed that such a conformational change is required for either activation or inhibition of the enzyme. The information obtained from this discovery has enabled identification of key interactions in the active site of the enzyme, as well as the design and opimization of inhibitors.

### CROSS-REFERENCE TO RELATED APPLICATION

This application claims the benefit of U.S. Provisional Application No. 60/301,676, filed Jun. 28, 2001, which is hereby incorporated by reference in its entirety.

### BACKGROUND OF THE INVENTION

This invention relates to new drug discovery methods, particularly methods of discovering new drugs that inhibit D-Ala-D-Ala ligase, an essential enzyme in the formation of bacterial cell walls.

Compounds that inhibit bacterial cell wall biosynthesis have generally been proven to be effective antibiotic agents. For example, the racemase inhibitor fluoro-D-alanine, which prevents the formation of D-alanine, and P-lactam antibiotics, which inhibit transpeptidation, inhibit cell wall synthesis and bacterial growth (Parsons et al., J. Med. Chem., 31:1772-1778, 1988). However, the recent emergence of drug resistant bacterial strains suggests there exists an ongoing need for new broad-spectrum antibiotics.

Among the enzymes responsible for cell wall biosynthesis, D-alanyl-D-alanine ligase (“D-Ala-D-Ala ligase”; E.C. 6.3.2.4) is important because it synthesizes the unique dipeptide D-alanyl-D-alanine (“D-Ala-D-Ala”). The dipeptide is ultimately incorporated into individual peptidoglycan strands, in which it provides the site for transacylation during peptidoglycan crosslinking, the final step of cell wall synthesis (Ellsworth et al., Chemistry & Biology, 3:37-44, 1996).

Inhibitors that prevent the assembly and incorporation of D-Ala-D-Ala into the cell wall are hypothesized to be effective antibiotics because they can cause bacterial lysis. D-Ala-D-Ala ligase inhibitors can be highly selective broad-spectrum antibiotics with relatively few adverse side effects, because D-Ala-D-Ala ligase is highly conserved among prokaryotes and is not present in humans. D-Ala-D-Ala ligase is a multi-domain protein that contains two binding pockets, one for ATP and another for D-Ala-D-Ala. Thus far, no useful inhibitors have been identified that bind to the ATP binding site of D-Ala-D-Ala ligase.

### SUMMARY OF THE INVENTION

The invention is based in part on the discovery that certain small molecules can bind to the ATP binding site of D-Ala-D-Ala ligase, even in the absence of the enzyme's substrate, and can cause a conformational change in the enzyme structure similar to that that occurs upon binding of ATP and substrate to the enzyme. Without wishing to be bound by any theory, it is believed that such a conformational change is required for either activation or inhibition of the enzyme. The information obtained from this discovery has enabled identification of key interactions in the active site of the enzyme, as well as the design and optimization of inhibitors.

In one embodiment, the invention features a method for evaluating the potential of a chemical entity to associate with a molecule or molecular complex comprising a binding pocket defined by structural coordinates of D-Ala-D-Ala ligase E. coli amino acids Lys144, Glu180, Lys181, Leu183, Glu187, Asp257, and Glu270 according to FIG. 8; or a homolog of said molecule or molecular complex, wherein said homolog comprises a binding pocket that has a root mean square deviation from the backbone atoms of said amino acids of not more than 10 Å. The method includes one or more, and preferably all of the steps of (1) employing a predictive method (e.g., a computer program or other computational means) to perform a fitting operation between the chemical entity and a binding pocket defined by structural coordinates of D-Ala-D-Ala ligase E. coli amino acids Lys144, Glu180, Lys181, Leu183, Glu187, Asp257, and Glu270 +/− a root mean square deviation from the backbone atoms of said amino acids of not more than 10 Å; and (2) analyzing the results of said fitting operation to quantify the association between the chemical entity and the binding pocket.

In another embodiment, the invention features a method for identifying a potential inhibitor of D-Ala-D-Ala ligase. The method includes the steps of: (1) using the position or structure of Lys144, Glu180, Lys181, Leu183, Glu187, Asp257, and Glu270 of E. coli D-Ala-D-Ala ligase according to FIG. 8 (e.g., using the atomic coordinates these amino acids) +/− a root mean square deviation from the backbone atoms of said amino acids of not more than 10 Å, to generate a three-dimensional structure of the D-Ala-D-Ala ligase binding pocket; (2) employing said three-dimensional structure to design or select said potential inhibitor (e.g., to design or select an inhibitor that satisfies the requirements imposed by the pattern of physical interactions defined by the above amino acids and or other amino acids in the enzyme's co-substrate binding site, which interactions may be similar to a preselected or reference pattern of interactions such as the interactions that occur upon binding to D-alanine or another substrate or co-substrate to the enzyme). In a preferred embodiment, the method further includes one or both of: (3) synthesizing or obtaining said inhibitor; and (4) contacting said inhibitor with D-Ala-D-Ala ligase to determine the ability of said potential inhibitor to inhibit D-Ala-D-Ala. Optionally, the employing step can include designing a molecule that, if docked within said three-dimensional structure, would have a hydrogen bond donor between 2.4 and 3.5 Å from one or both carboxylate oxygen atoms of the Glu180 side chain, a hydrogen bond donor between 2.4 and 3.5 Å from the backbone amide oxygen of Lys181, a hydrogen bond acceptor between 2.4 and 3.5 Å from the backbone amide nitrogen of Leu183, a hydrogen bond donor between 2.74 and 3.5 Å from the backbone amide oxygen of Leu183, and a hydrogen bond acceptor between 2.4 and 3.5 Å from the side chain nitrogen of Lys144. The molecule can further include hydrophobic interactions 3.5-4.5 Å from the CD1 carbon and SD sulfur atoms of the side chains of Leu269 and Met154, respectively. The potential inhibitor can also be a bisubstrate analog (e.g., an analog that can bind to both the ATP-binding site and the D-Ala-binding site of the enzyme).

In still another embodiment, the invention features a method for identifying a potential inhibitor of D-Ala-D-Ala ligase or a homolog of D-Ala-D-Ala ligase. The method includes the steps of (1) designing or selecting a molecule that results in Ile142 of D-Ala-D-Ala ligase or its counterpart in a homolog being brought within 12 Å of Met259 of D-Ala-D-Ala ligase or its counterpart in a homolog, and Met154 of D-Ala-D-Ala ligase or its counterpart in a homolog being brought within 12 Å of Leu269; (2) synthesizing or obtaining said inhibitor; and (3) contacting said inhibitor with D-Ala-D-Ala ligase to determine the ability of said potential inhibitor to inhibit D-Ala-D-Ala.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a hypothetical structural drawing of a D-Ala-D-Ala ligase enzyme in the absence of substrates and/or cofactors, based on crystallographic data and showing the relative positions of the ATP- and D-Ala-D-Ala-binding sites and the four domains of the protein.

FIG. 2 is a superposition of the crystal structures of D-Ala-D-Ala ligase, complexed either with ATP alone, or with ADP, phosphate, and D-Ala-D-Ala, as shown in red and yellow, respectively. The arrow indicates the direction of the rigid body rotation of domain B in going from the former structure to the latter.

FIG. 3 is a series of schematics of the conformational change that is hypothesized to occur along the reaction pathway of the enzyme upon binding of ATP or an inhibitor to the ATP-binding site of D-Ala-D-Ala ligase. The schematics correspond to the unbound enzyme (E), a model of the initial inhibitor complex (EI), and the crystal structure of the enzyme after the inhibitor-induced conformational change (EI*).

FIG. 4 is a drawing that illustrates at least some of the key electrostatic (a) and hydrophobic (b) interactions between active-site residues of the enzyme and an inhibitor that induces a conformational change in the ligase. Dashed lines correspond to hydrogen bonds formed between conserved protein residues and the inhibitor. The residues shown in (b) participate in Van der Waals interactions with the inhibitor.

FIG. 5 is a graph of rate of stopped flow-ligase binding vs. ATP concentration.

FIG. 6 is a graph of fluorescence quenching of D-Ala-D-Ala ligase vs. ATP concentration.

FIG. 7 is an interaction map derived from a crystal structure of a new inhibitor bound to D-Ala-D-Ala ligase.

FIG. 8 is a list of the atomic structure coordinates for E. coli D-Ala-D-Ala ligase in complex with ADP, phosphate ion, and D-Ala-D-Ala as derived by X-ray diffraction from a crystal of that complex.

FIG. 9 is a list of the atomic structure coordinates for E. coli D-Ala-D-Ala ligase in complex with AMPPNP as derived by X-ray diffraction from a crystal of that complex.

FIG. 10 is a table of alignment data for fifty-one D-Ala-D-Ala ligase sequences from different strains of bacteria.

### DETAILED DESCRIPTION OF THE INVENTION

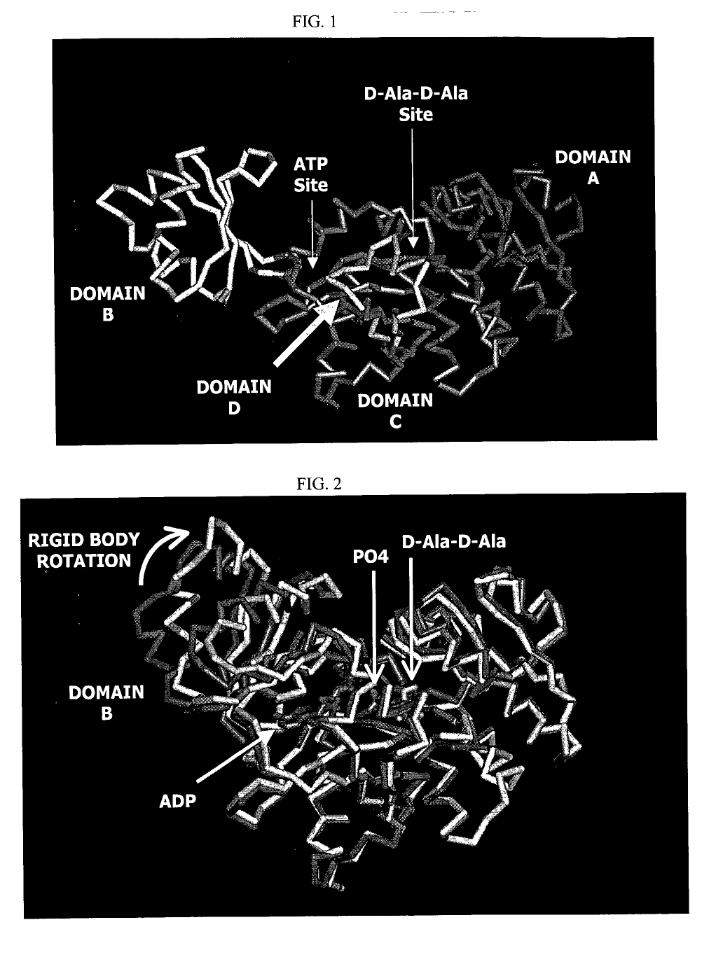

Characterization of the conformational change D-Ala-D-Ala ligase is a multi-domain protein consisting of four domains, whose interfaces create the D-Ala-D-Ala and ATP binding pockets (FIG. 1). The conformational change was observed by determining the crystal structure of the enzyme in complex with ligands that are competitive inhibitors of ATP; biochemical assays confirmed the existence of the conformation change using two kinetic assays.

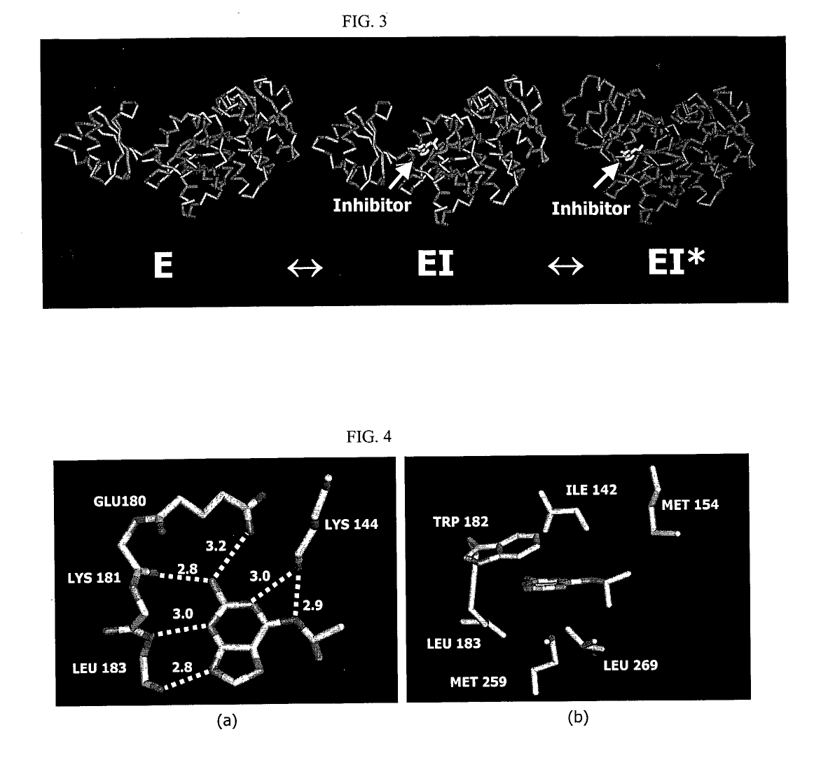

Structural methods for identifying the conformational change The conformational flexibility of the enzyme was first identified by comparing two crystal structures: that of (1) the enzyme in complex with ATP (EI*) and (2) the enzyme in complex with ADP, phosphate, and D-Ala-D-Ala (EP). A superposition of the two structures reveals a slight rigid body rotation of domain B into the active site when the enzyme is complexed with ADP, phosphate, and D-Ala-D-Ala (FIG. 2). This result suggests that the hinge point connecting domain B is fairly flexible and that domain B likely undergoes a significant rigid body movement when ligands bind between at the interface of domains B and C. An illustration of the sequence of events that takes place when ligands first bind to the enzyme and the potential magnitude of the induced conformational change is shown in FIG. 3, where EI is a hypothesized initial complex.

Stopped flow studies on ligase

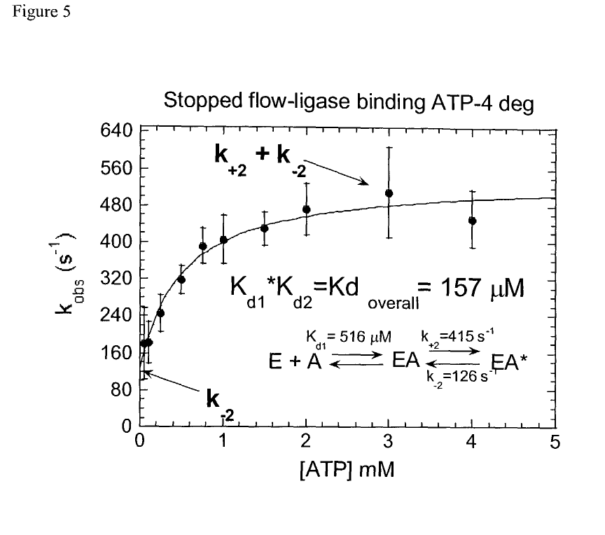

We have discovered a significant fluorescence quenching upon binding of ATP and ADP, which we have exploited to examine mechanistic features of ligase. We have carried out stopped flow studies to look at the binding of ATP and ADP to ligase. These studies were carried out at 4° C. We observe a single exponential fluorescent quenching which is completed in <20 ms. The observed rate constants plotted as a function of nucleotide concentration yield a hyperbolic plot indicating that the initial binding is followed by a conformational change (FIG. 5). This confirms our previous hypothesis about ligase, namely that the enzyme undergoes conformational changes that are an important and integral part of its enzymatic mechanism. This enzyme appears to fall into the category of “induced fit”.

As shown in FIG. 5, the initial collision complex is relatively weak to form the EA complex (open complex). The enzyme undergoes a conformational change to form the partially closed complex EA*. For ATP, this conformational change increases the affinity by 3.2 fold to a final K_{d}=157 μM (the overall affinity is the product of the two dissociation constants K_{d1} and K_{d2}), with a net dissociation rate constant of 126 s⁻¹. ADP exhibits a similar hyperbolic dependence, again indicative of an induced fit mechanism (i.e. a conformational change following binding). For ADP the conformational change increases the affinity of the nucleotide seven-fold for the partially closed complex, with respect to the initial collision complex, leading to an overall K_{d} of 50 μM. We hypothesize that making more interactions can increase the affinity, and hence stabilize this partially closed form. To dissociate the ligand, the enzyme has to relax back to the open form. Hence, the affinity of these inhibitors probably correlates with a decrease in the net dissociation rate constant (i.e., k₋₂). For example, ADP has a three-fold higher affinity than ATP does for D-Ala-D-Ala ligase, and has a slower k₋₂=72 s⁻¹. In some cases, it can be advantageous for the inhibitor to trigger a further conformational change, perhaps the closure of the omega loop of domain D, leading to a fully closed form of the enzyme.

Stopped flow studies have added to the understanding of the mechanism by which ligase binds ligands, and have confirmed previous suspicions about “induced fit” mechanism. Determining the affinity of high affinity inhibitors (low nM) will be difficult by equilibrium binding methods or steady state enzyme kinetics. Stopped flow studies may well be the only way that the affinity of high affinity inhibitors can be determined with any degree of confidence. The studies can be carried out, for example, using the methods described by Eccleston, J. F. “Stopped-flow Spectrophotometric Techniques” in Spectrophotometry and Spectrofluorimetry a Practical Approach, Ed. D. A. Harris & C. L. Bashford, IRL Press, 1987, p. 137-164.

Fluorescent Titration Experiments

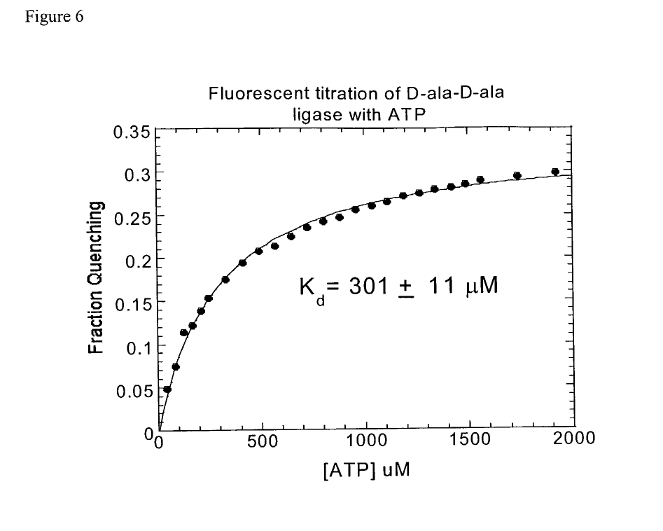

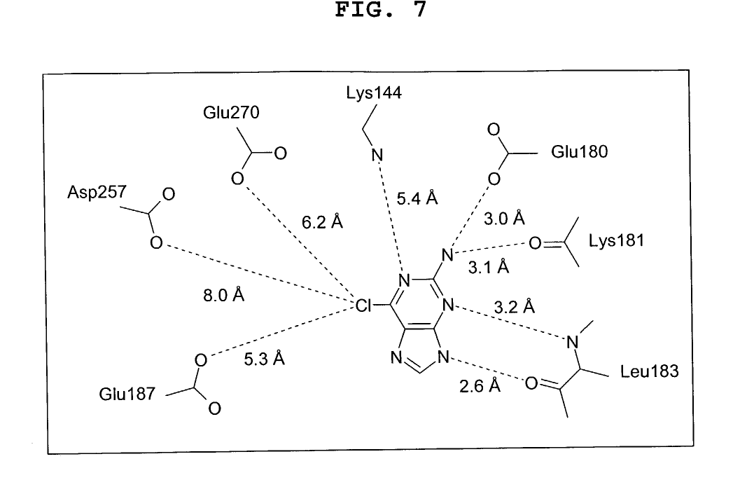

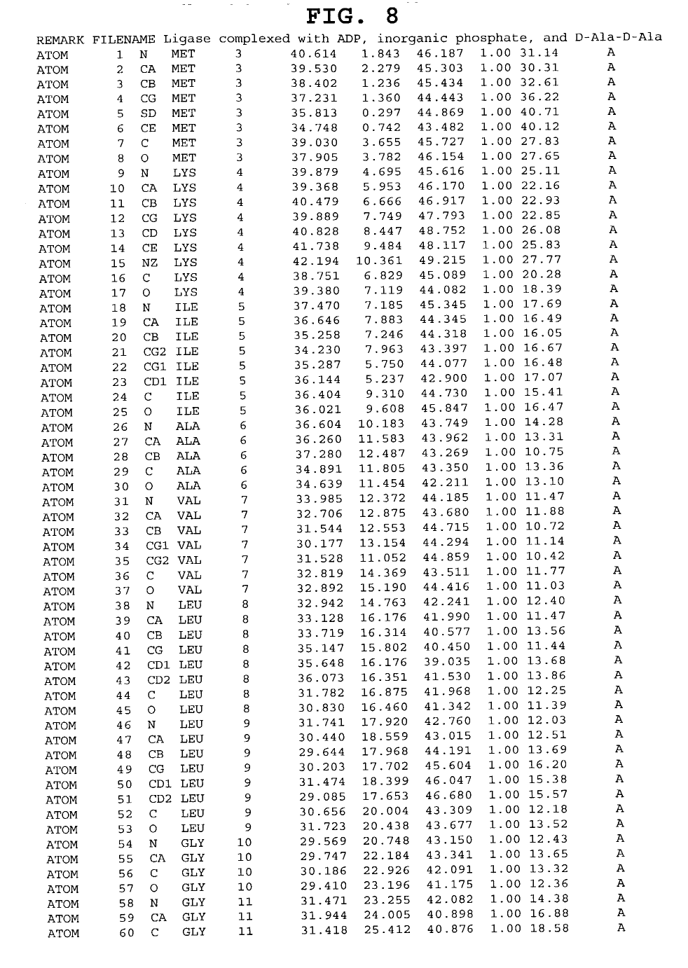

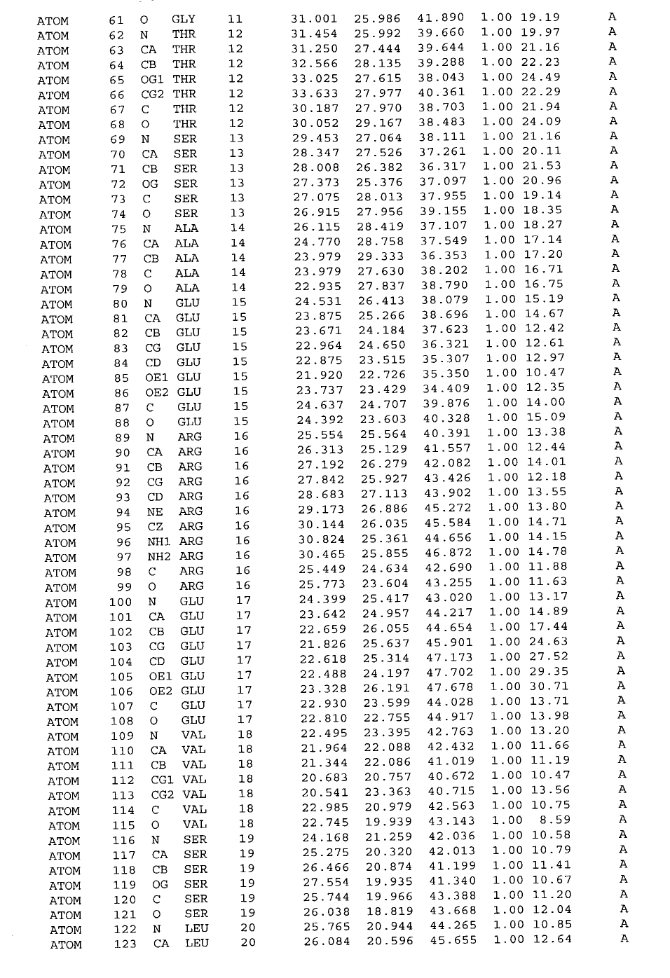

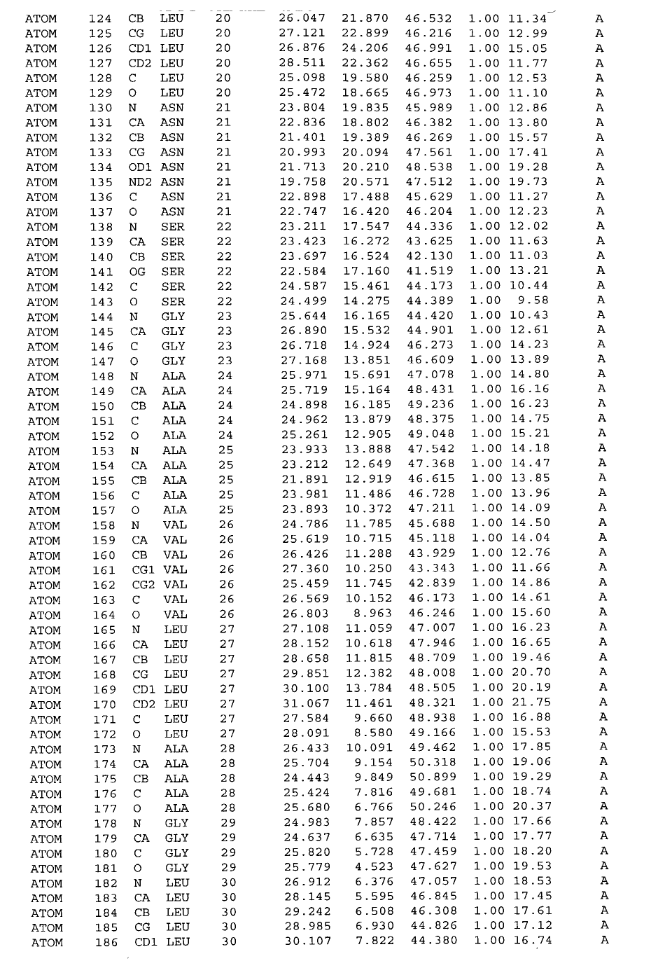

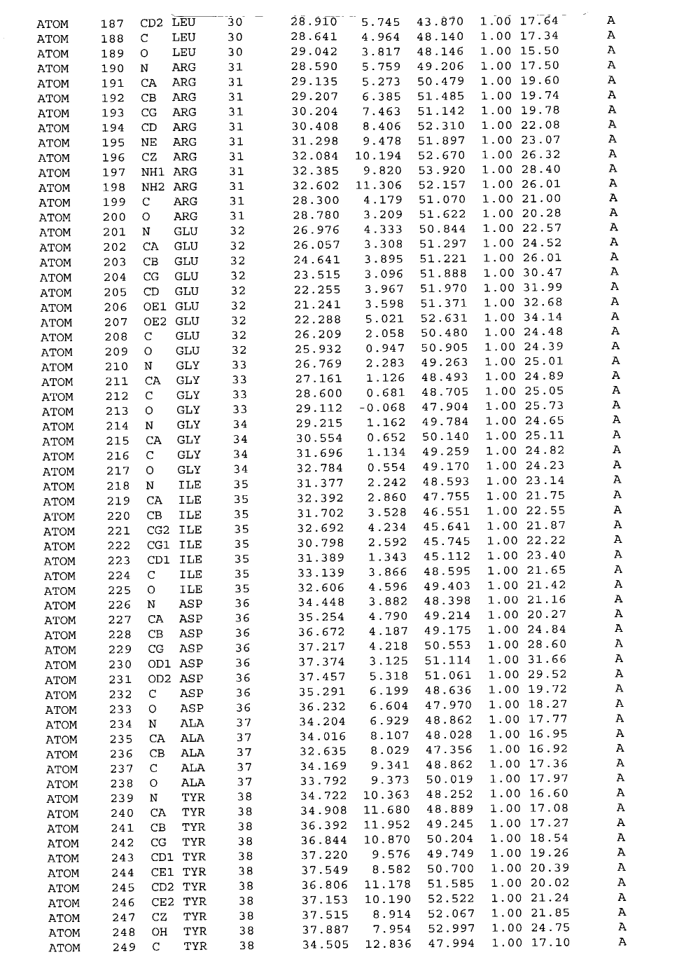

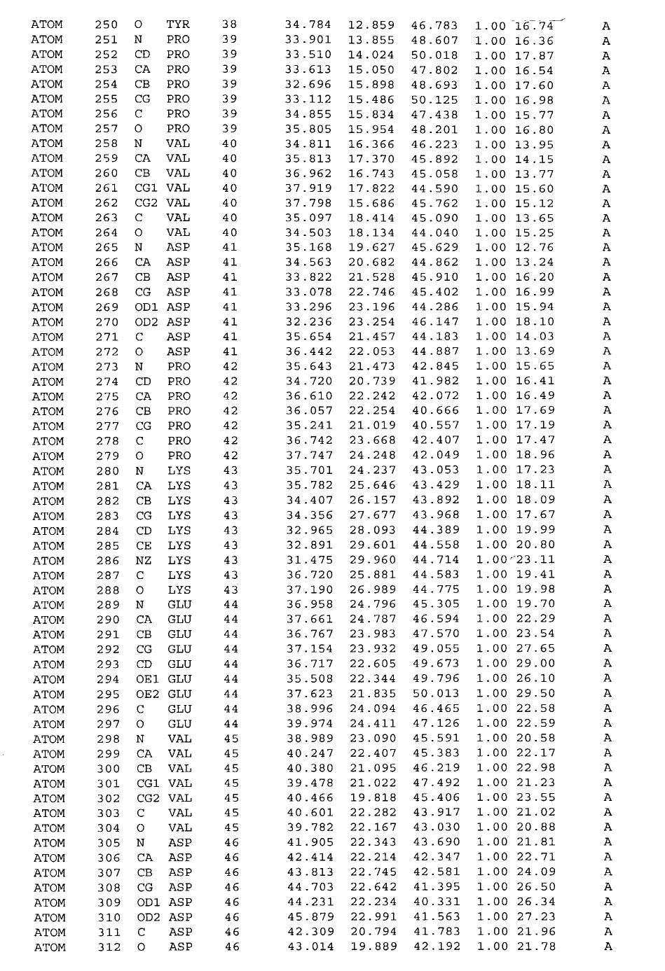

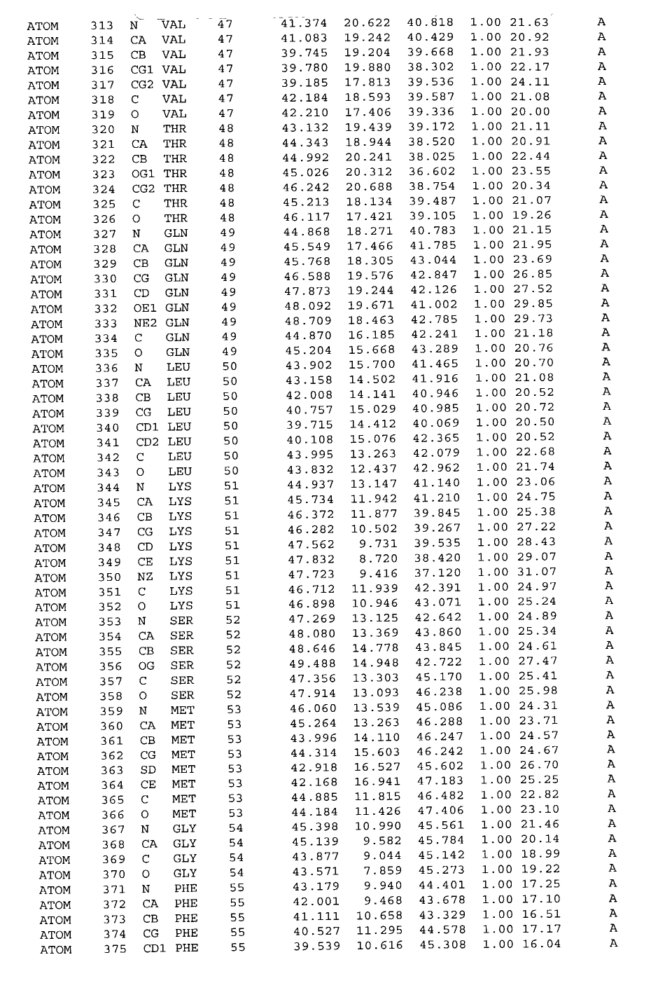

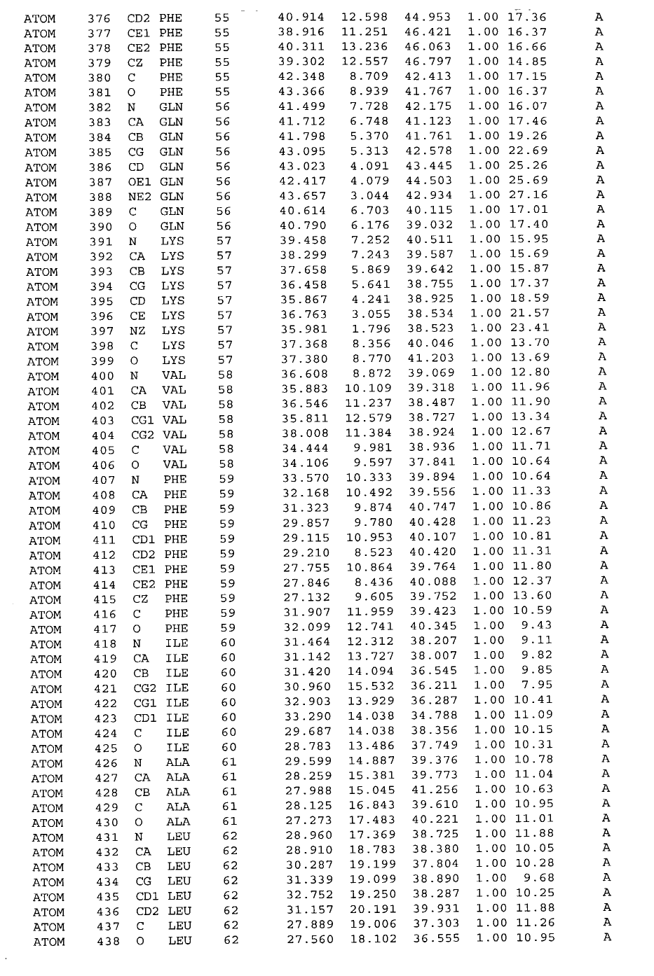

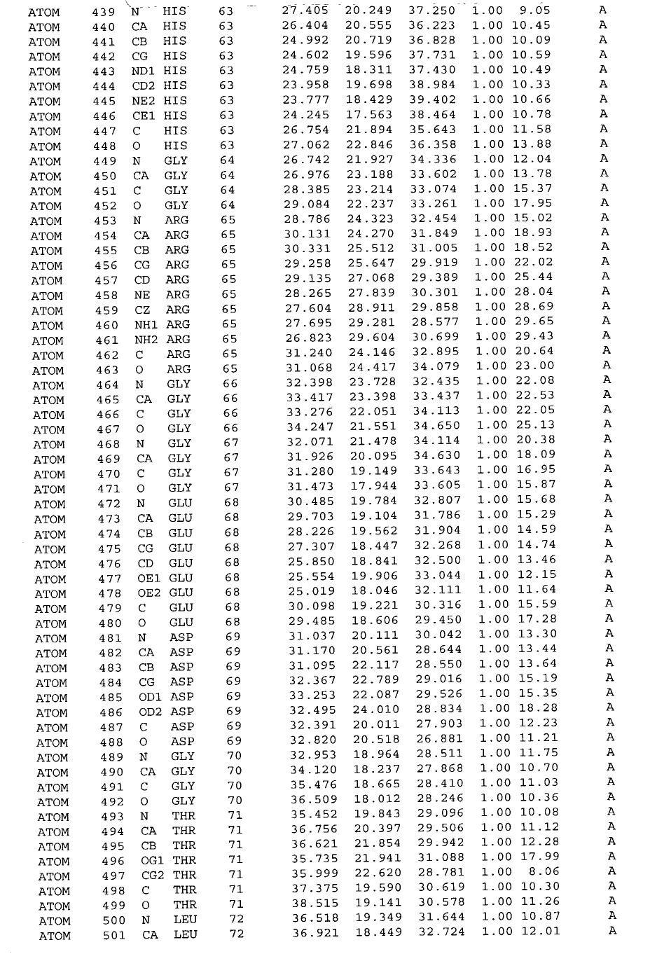

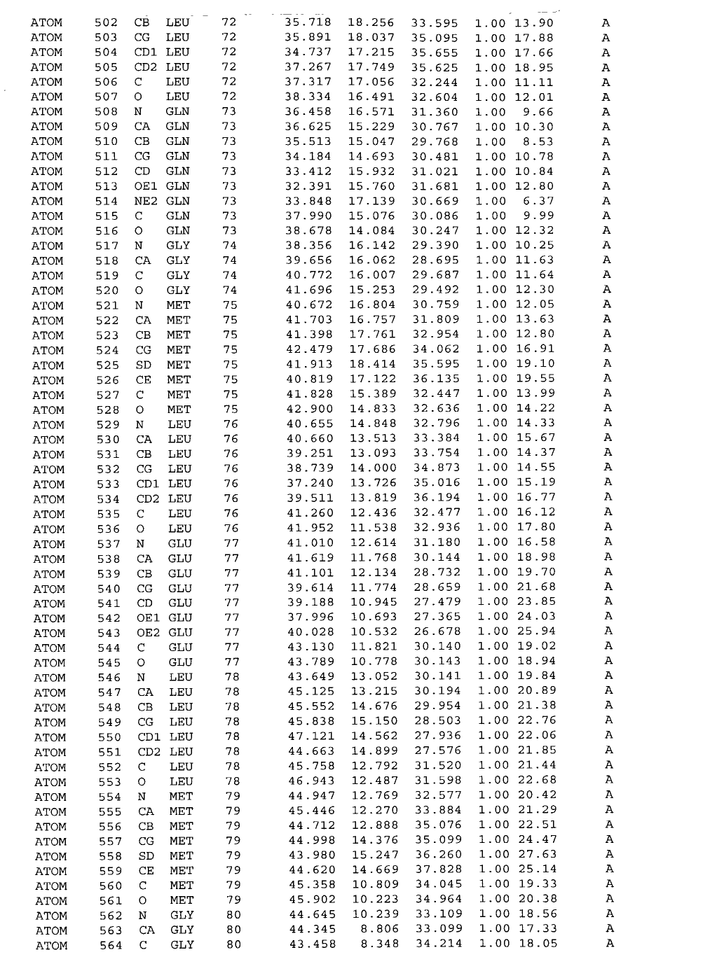

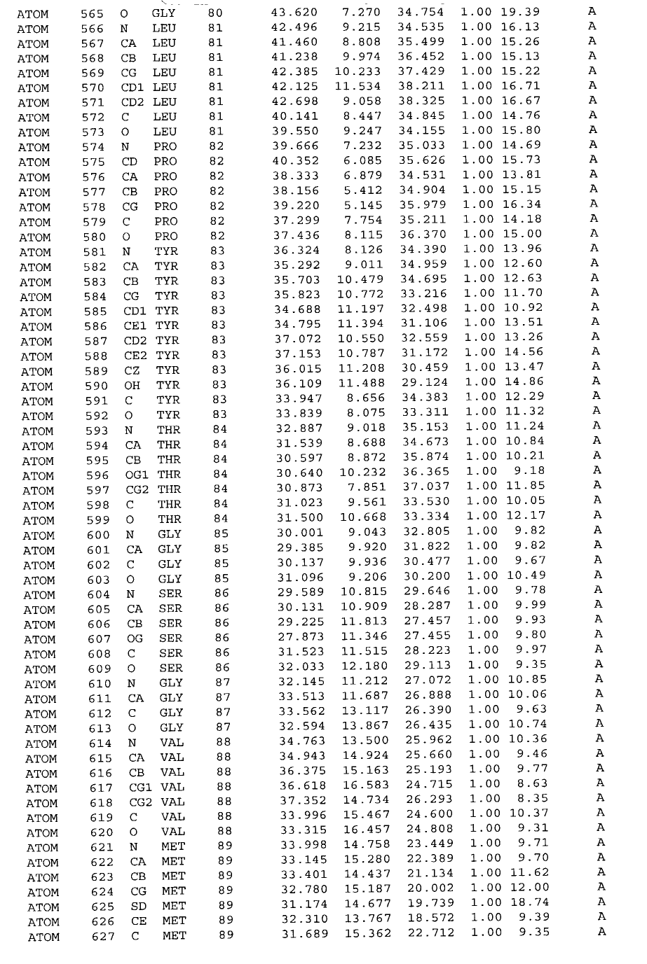

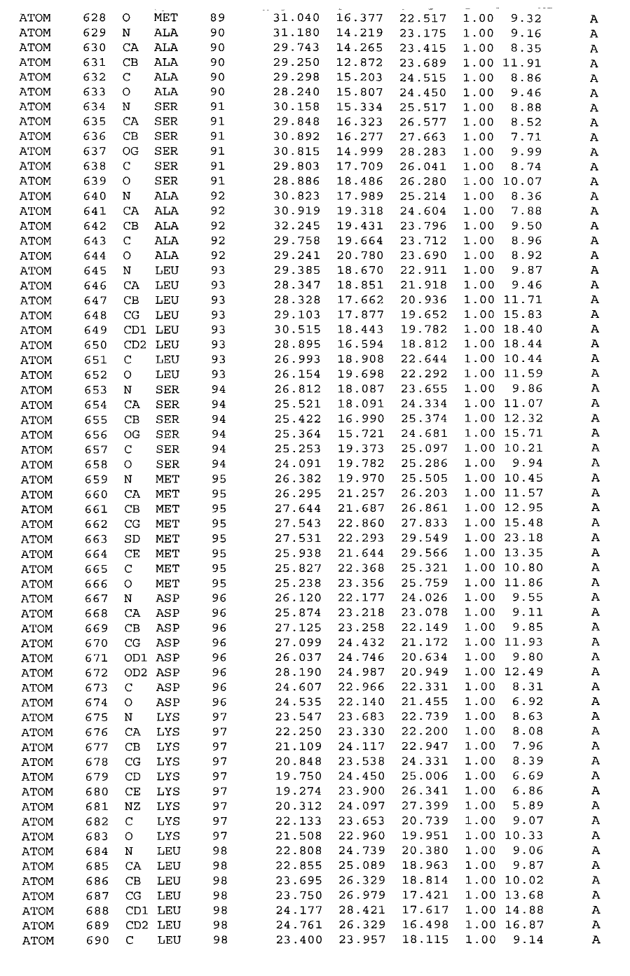

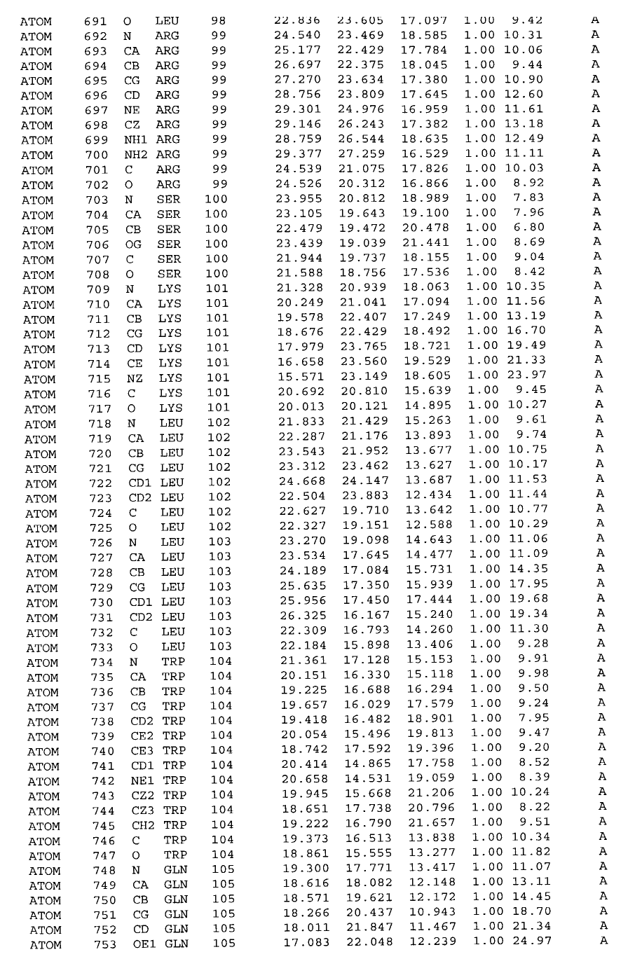

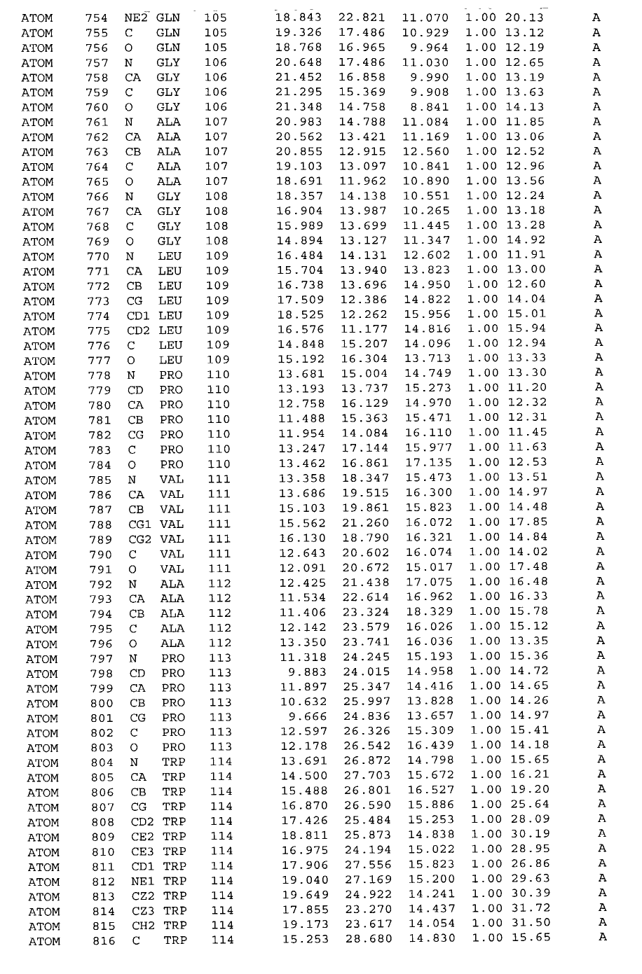

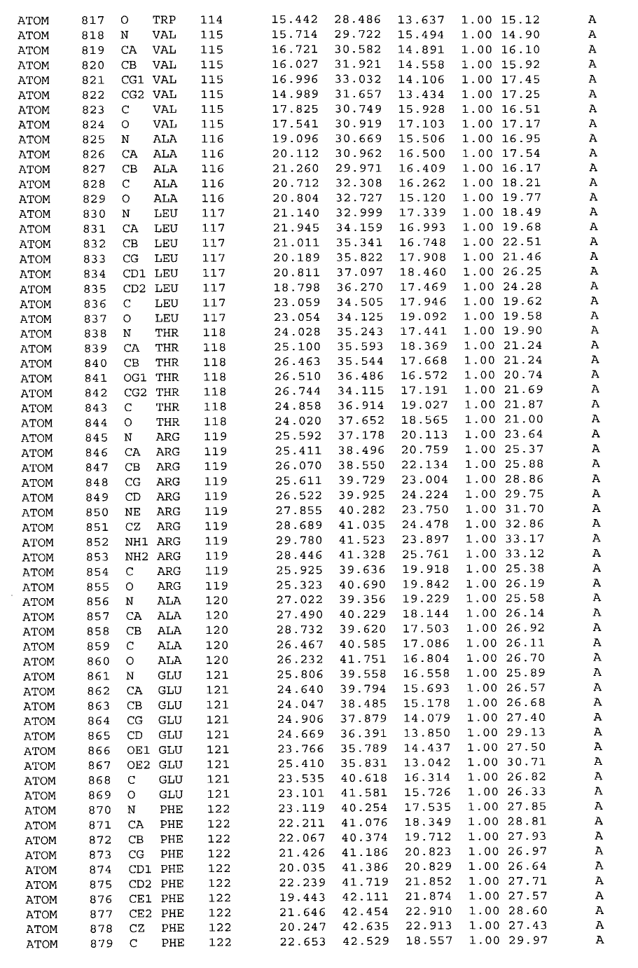

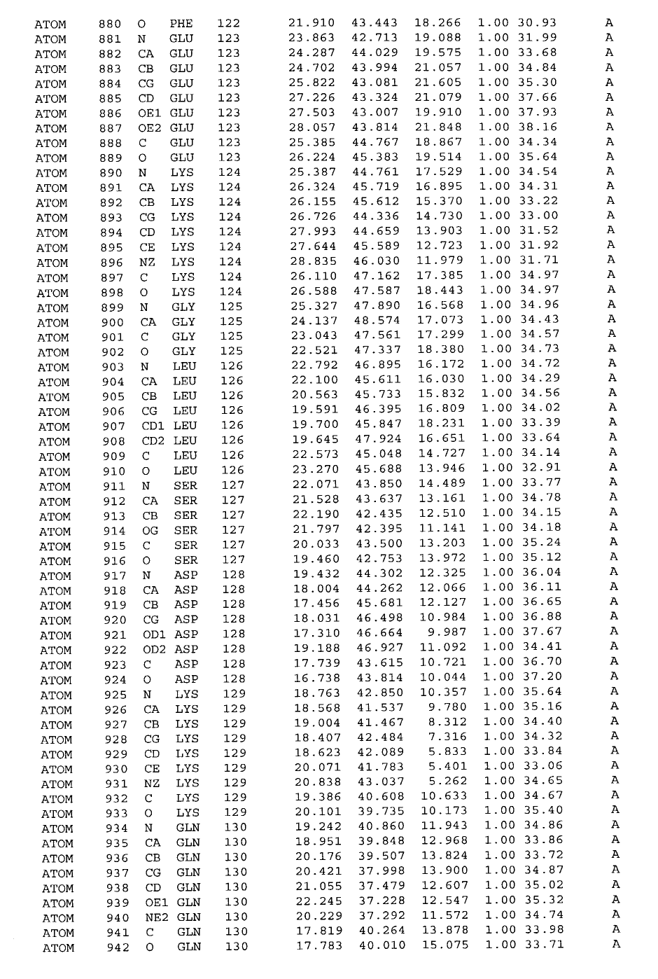

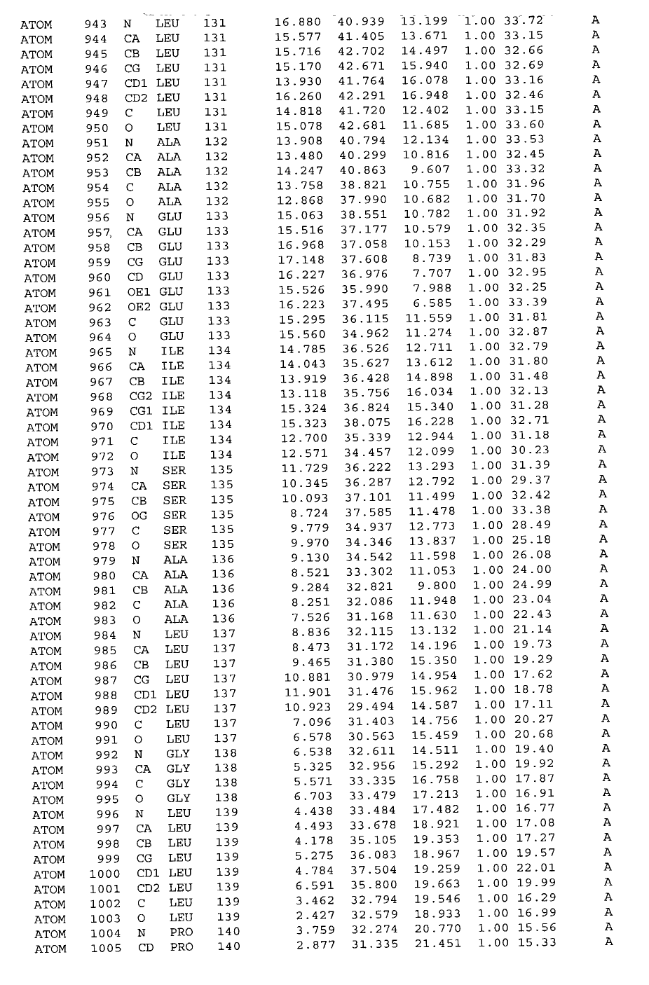

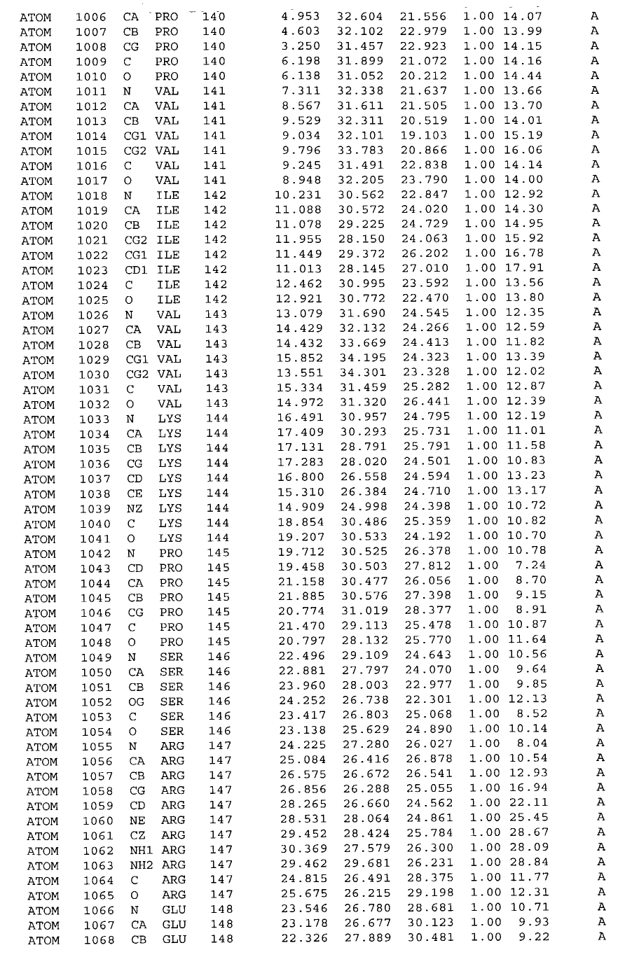

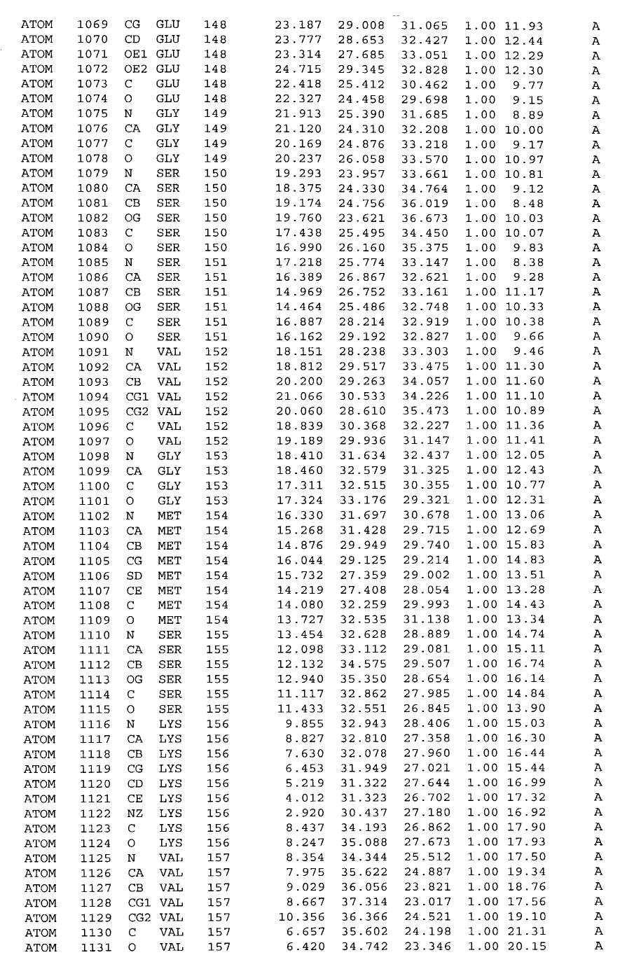

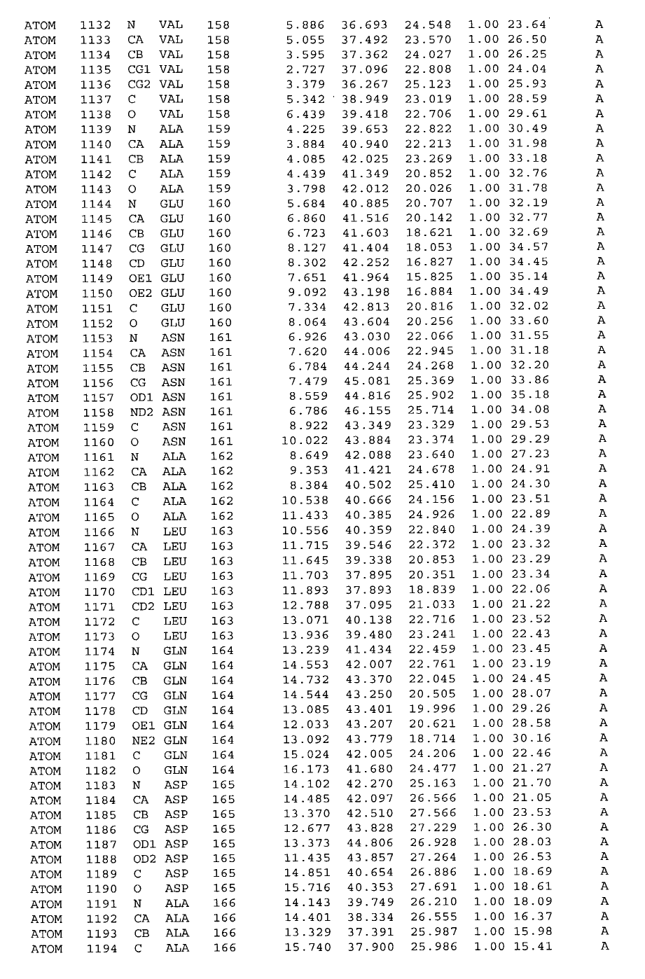

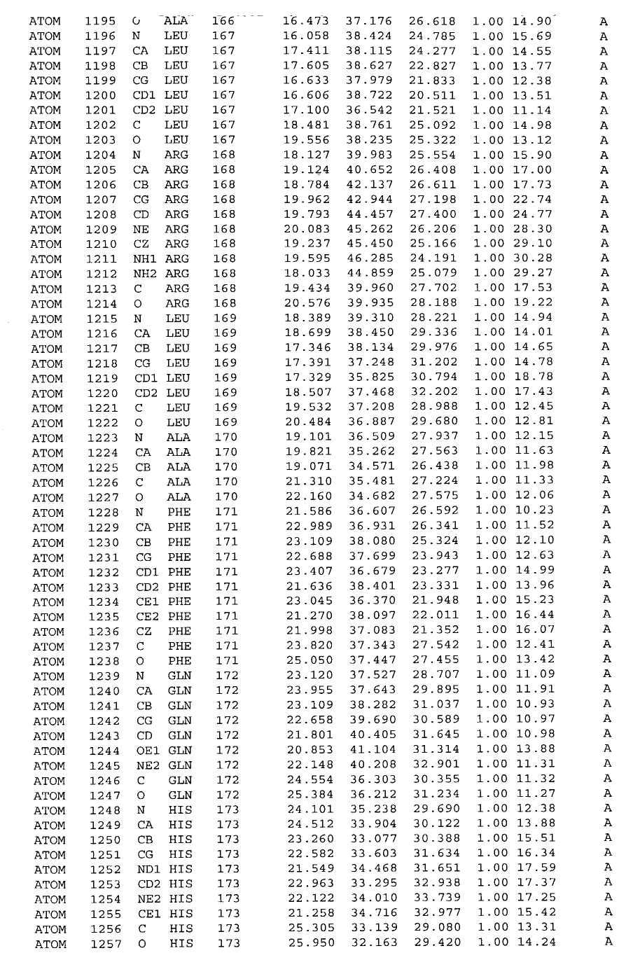

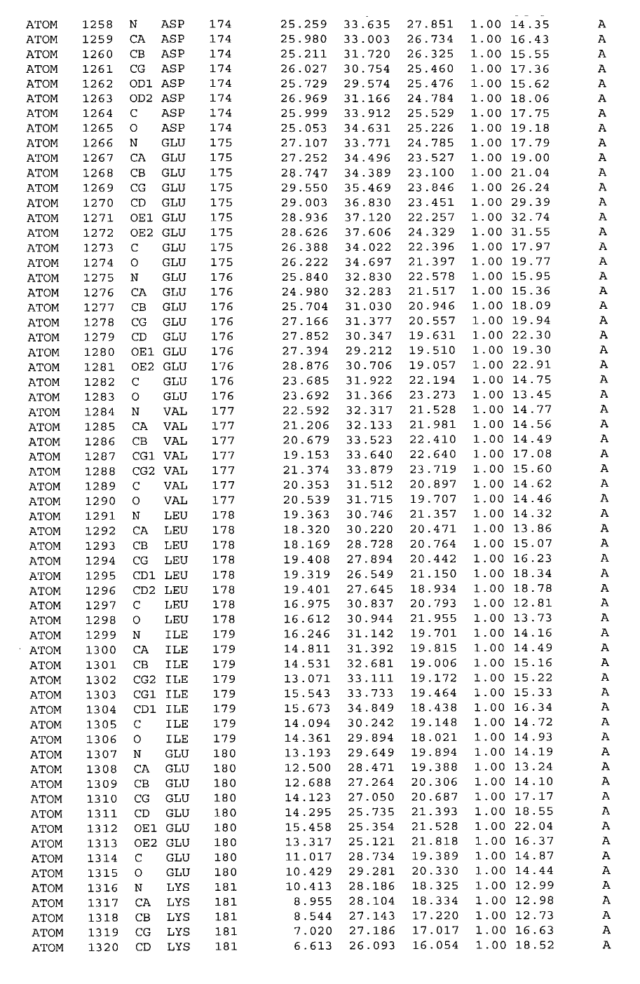

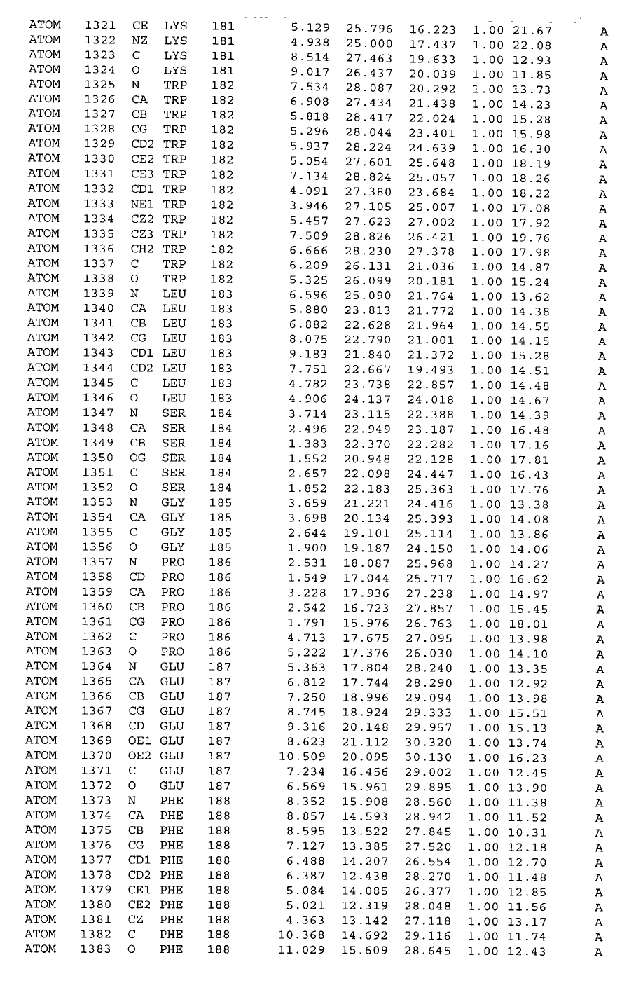

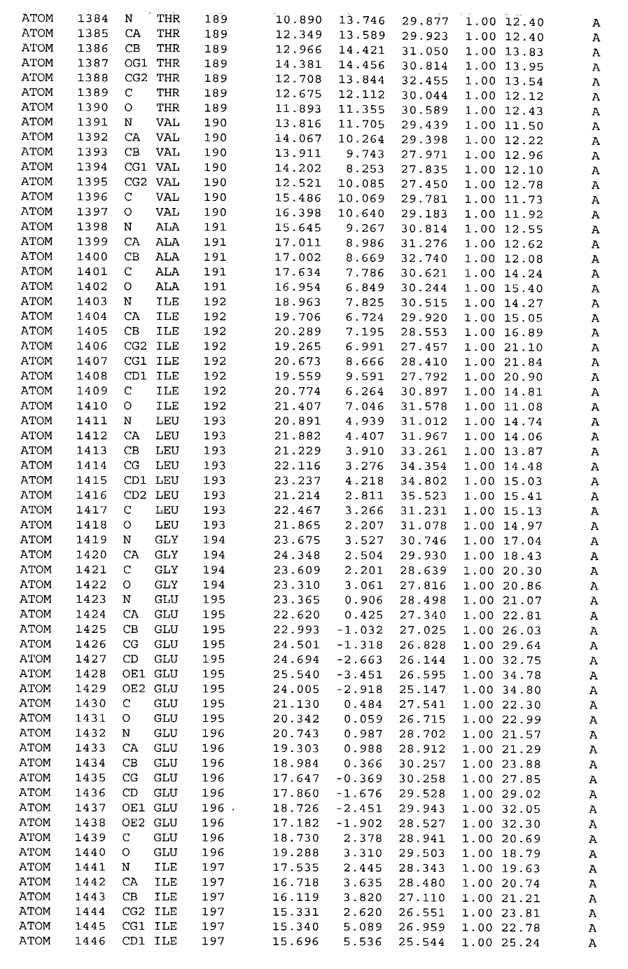

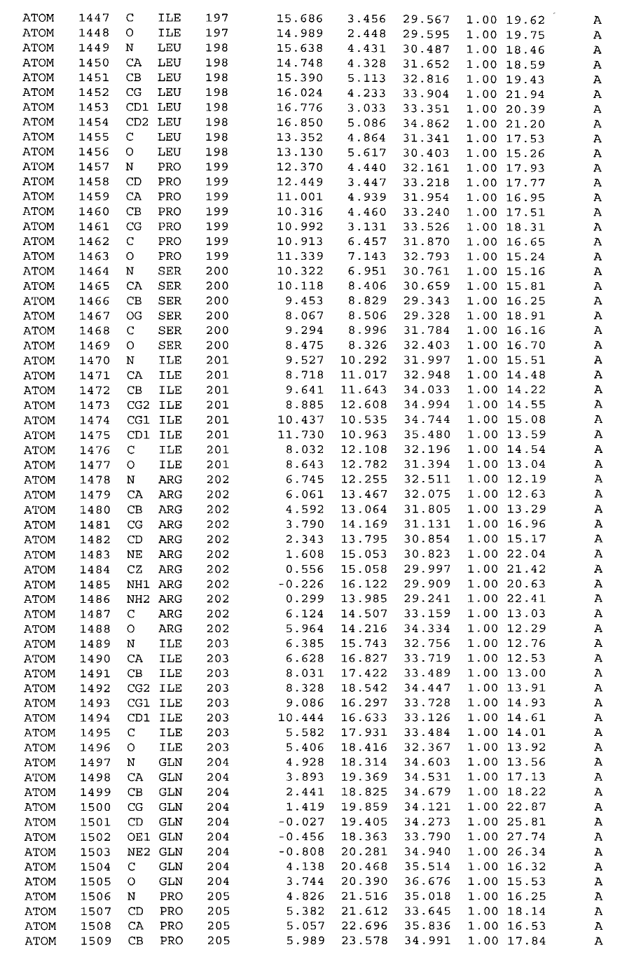

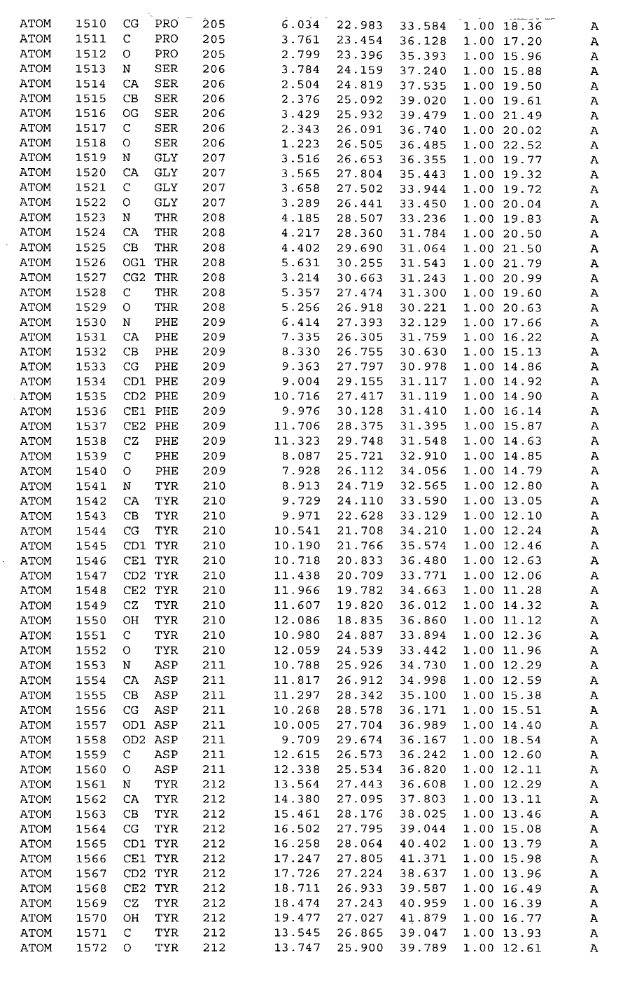

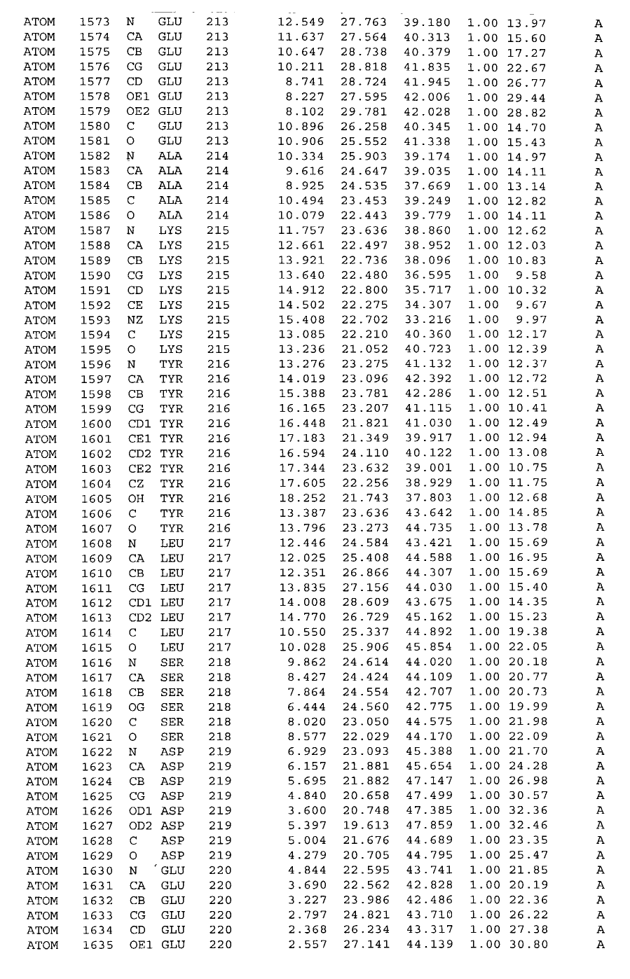

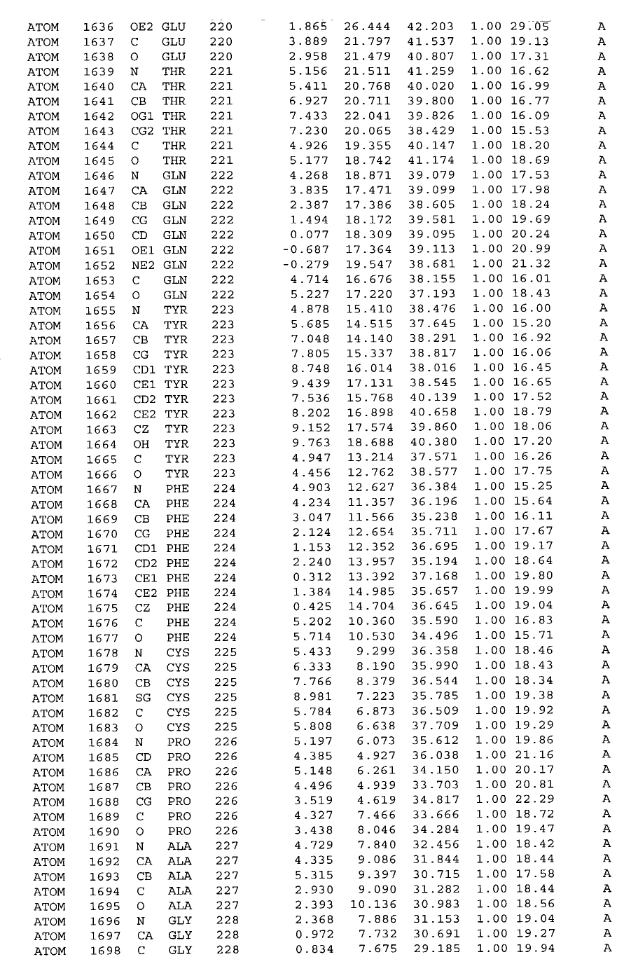

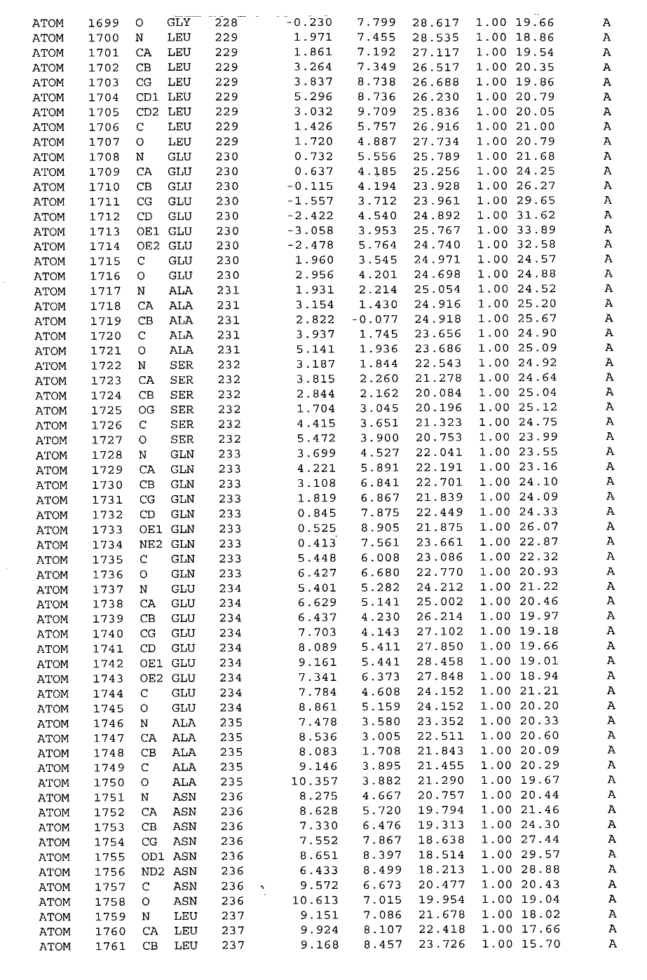

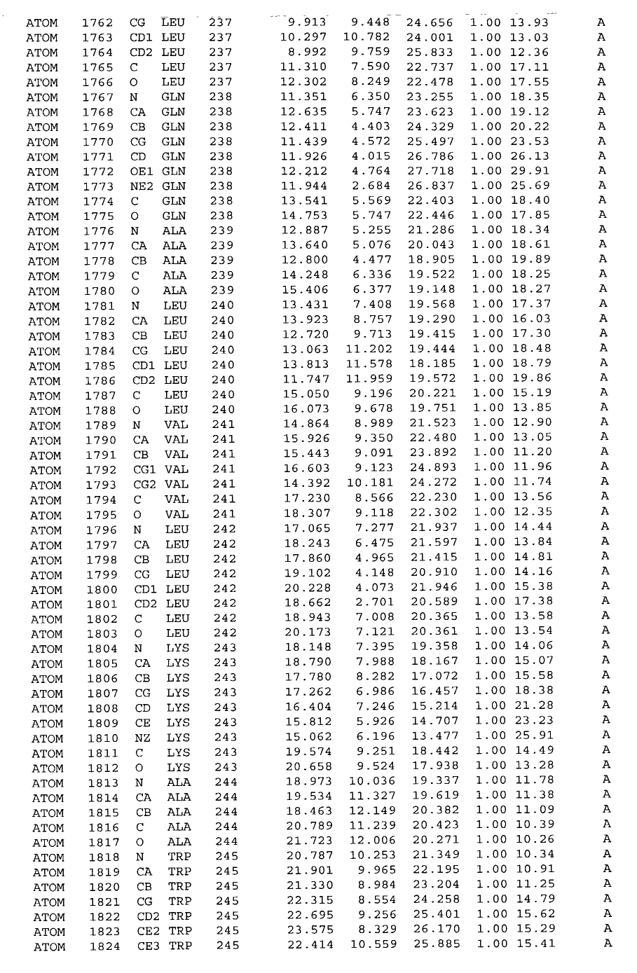

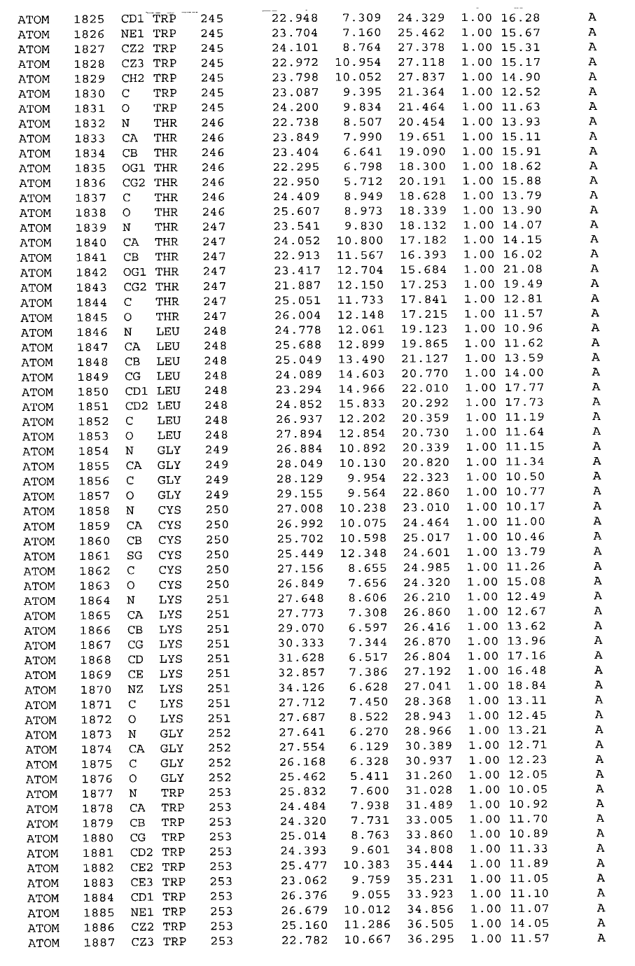

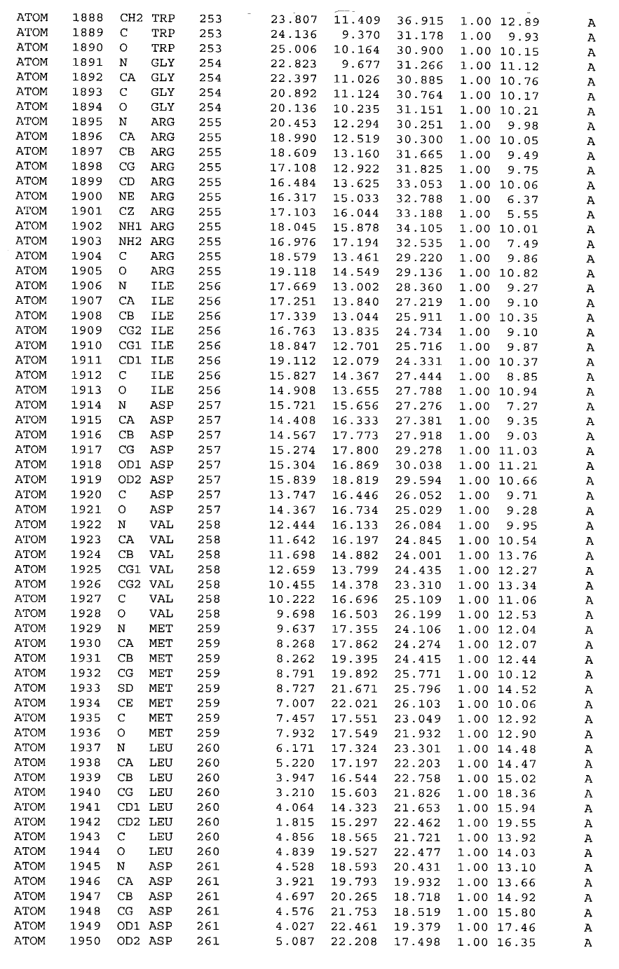

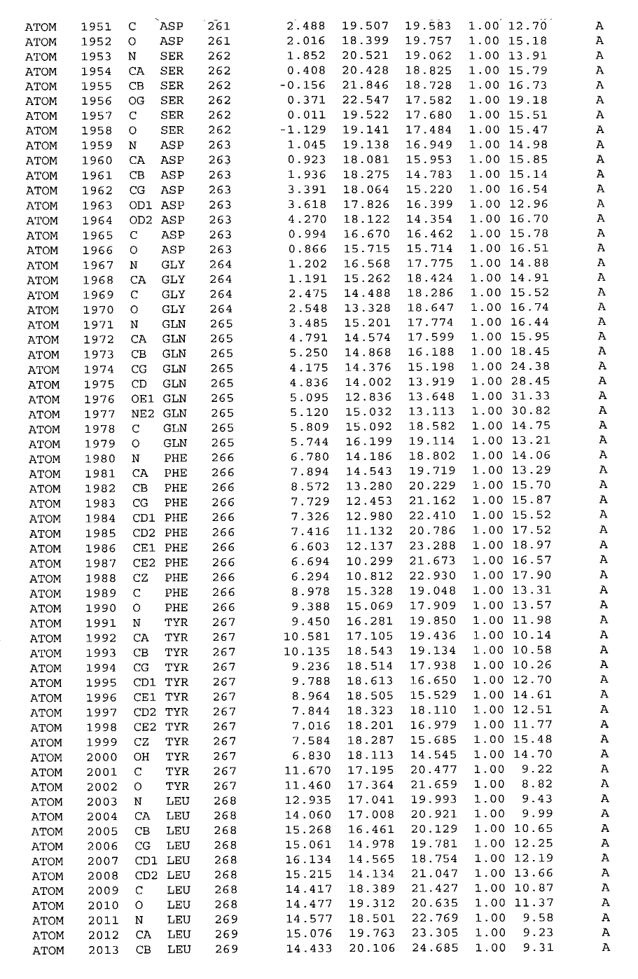

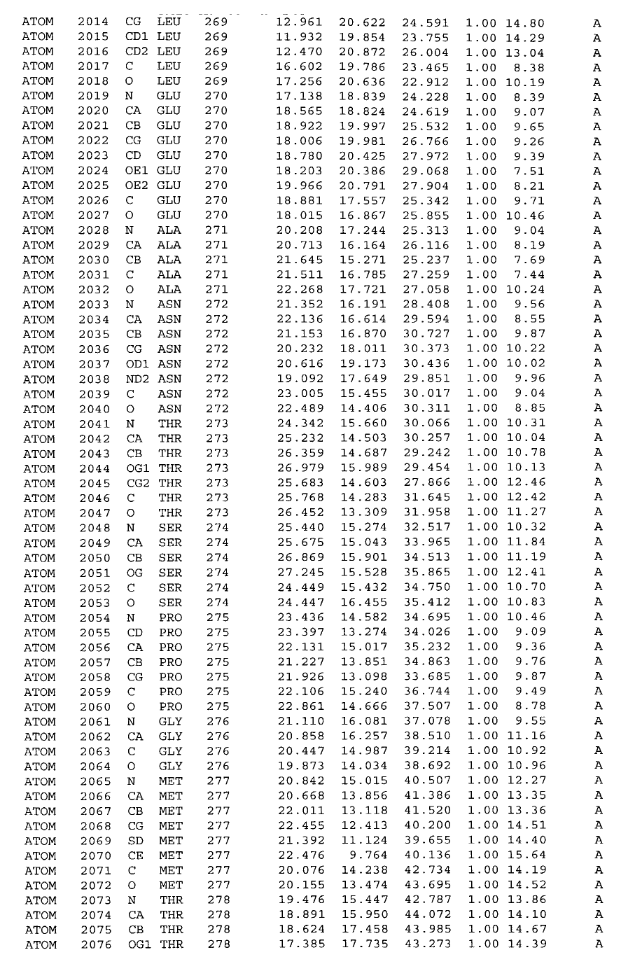

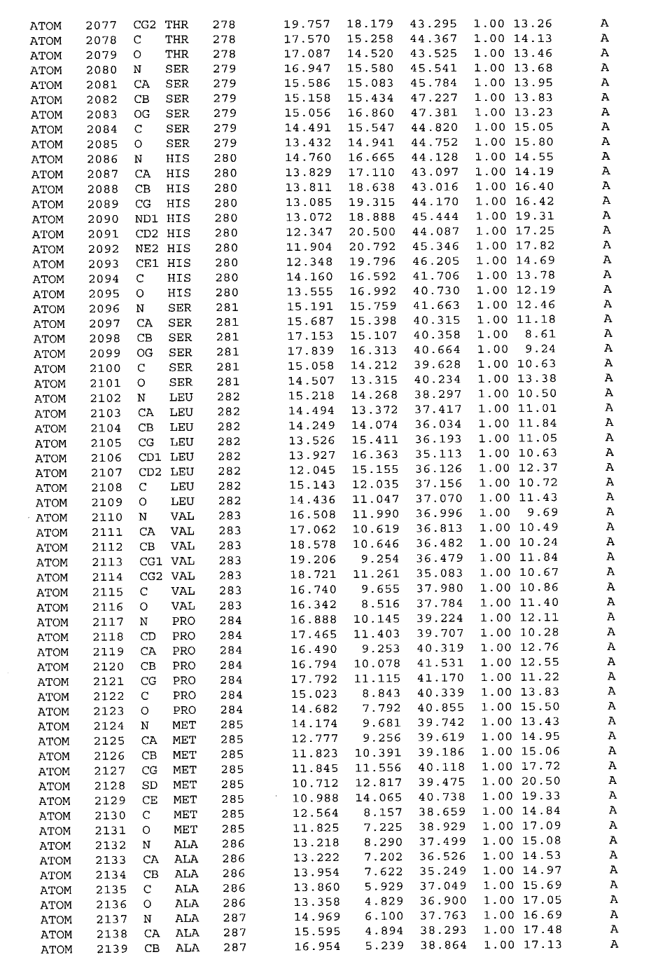

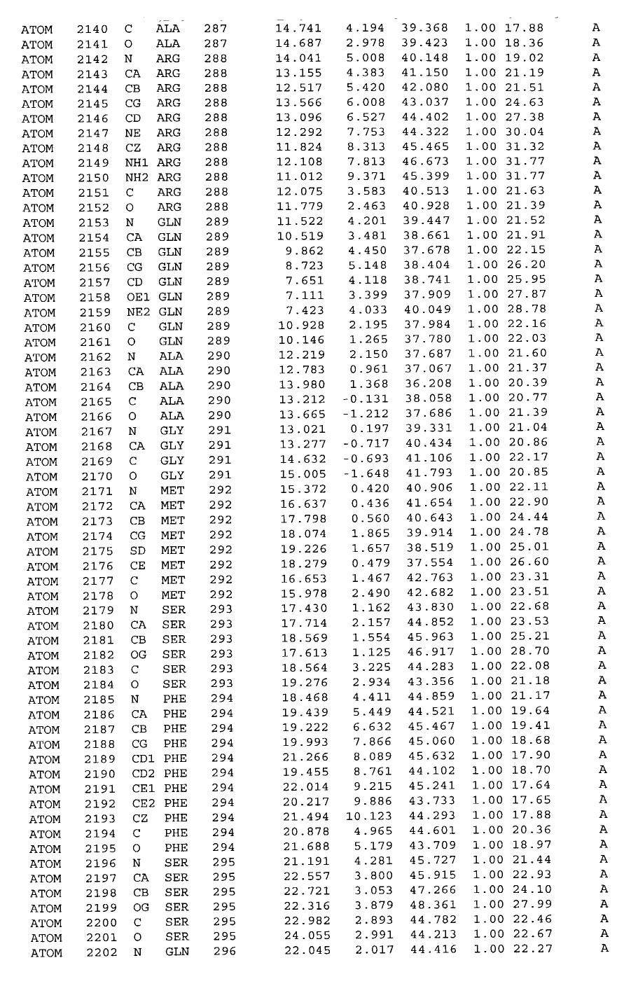

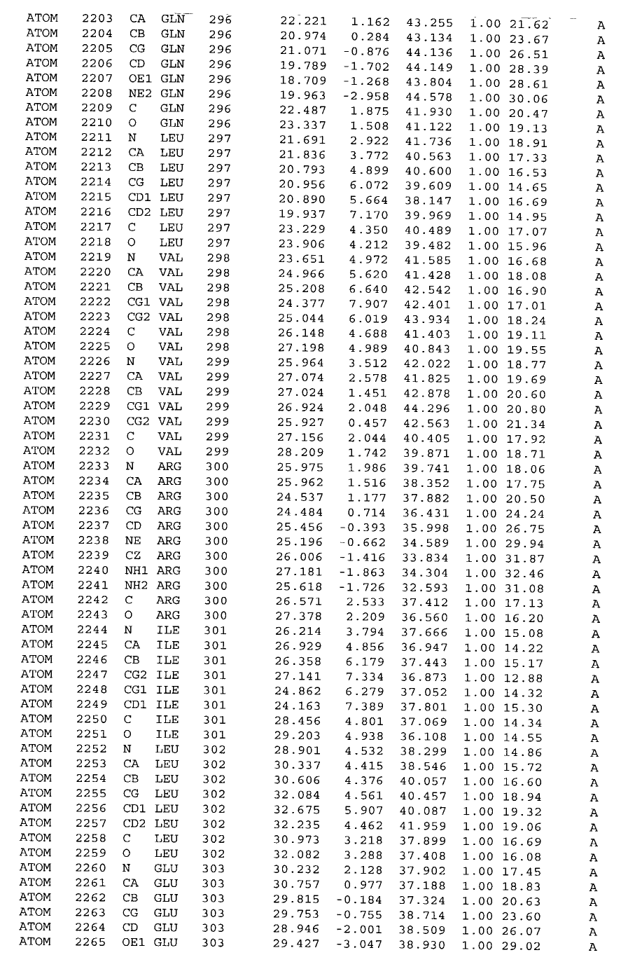

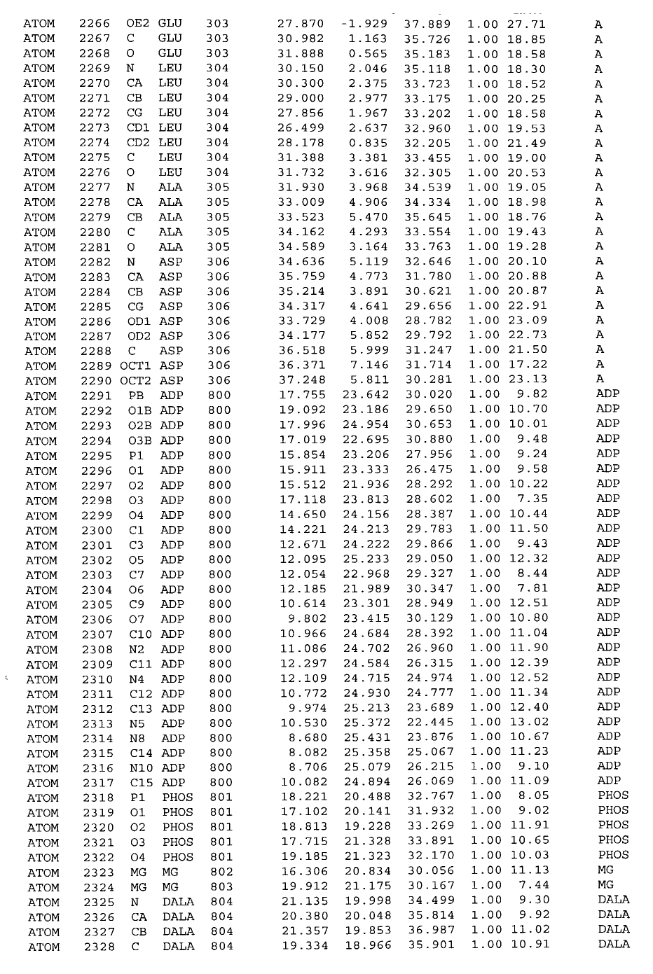

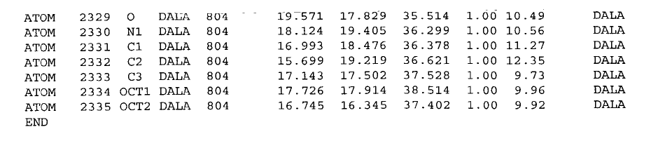

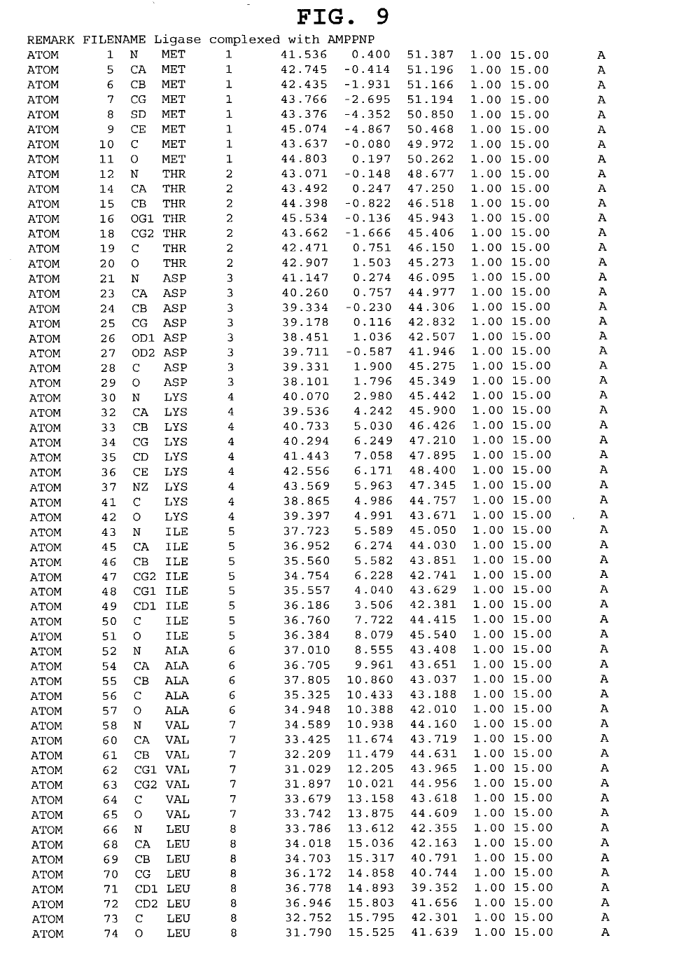

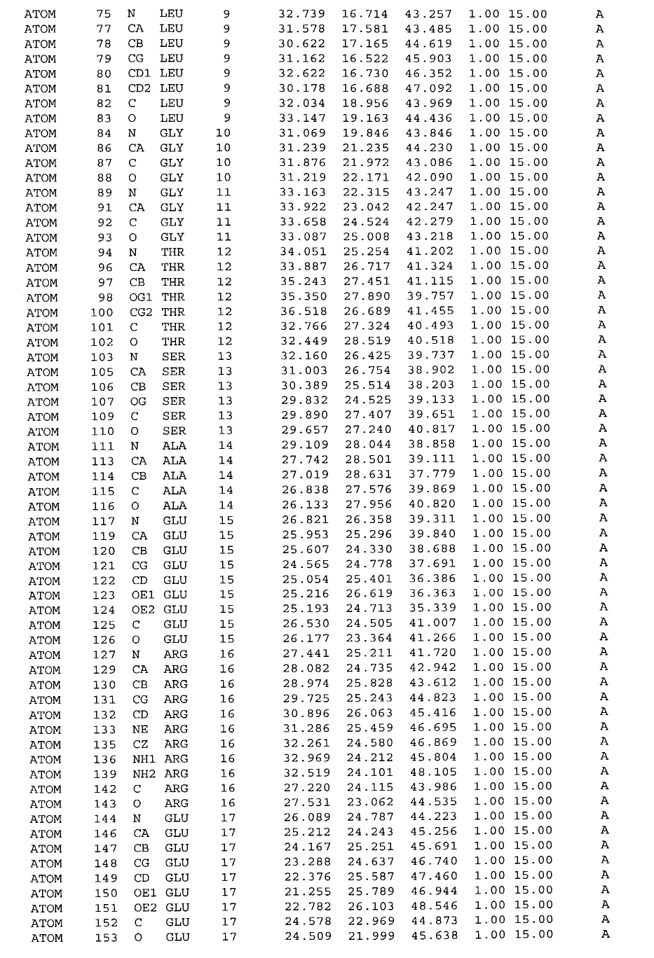

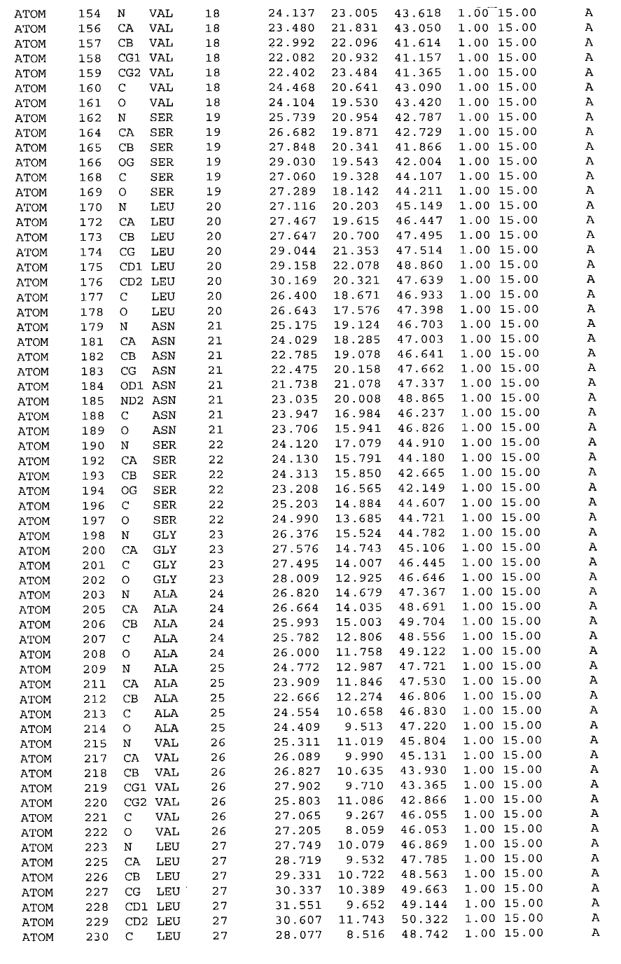

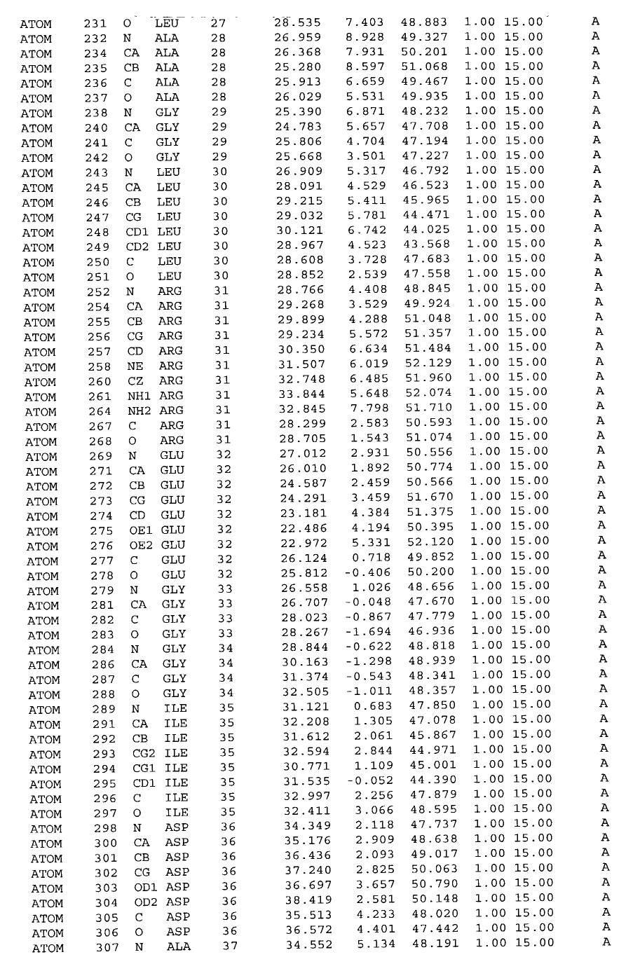

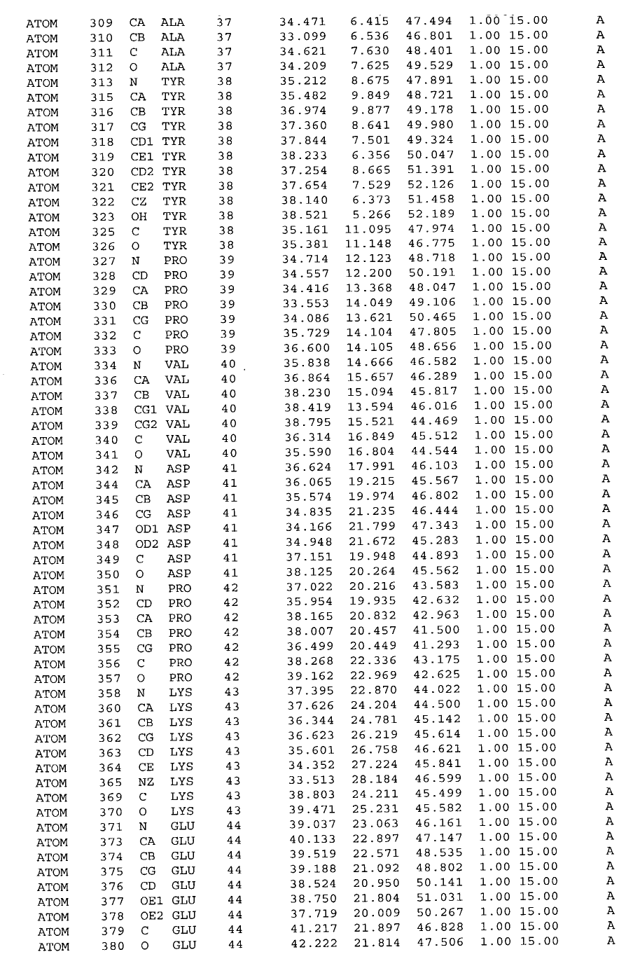

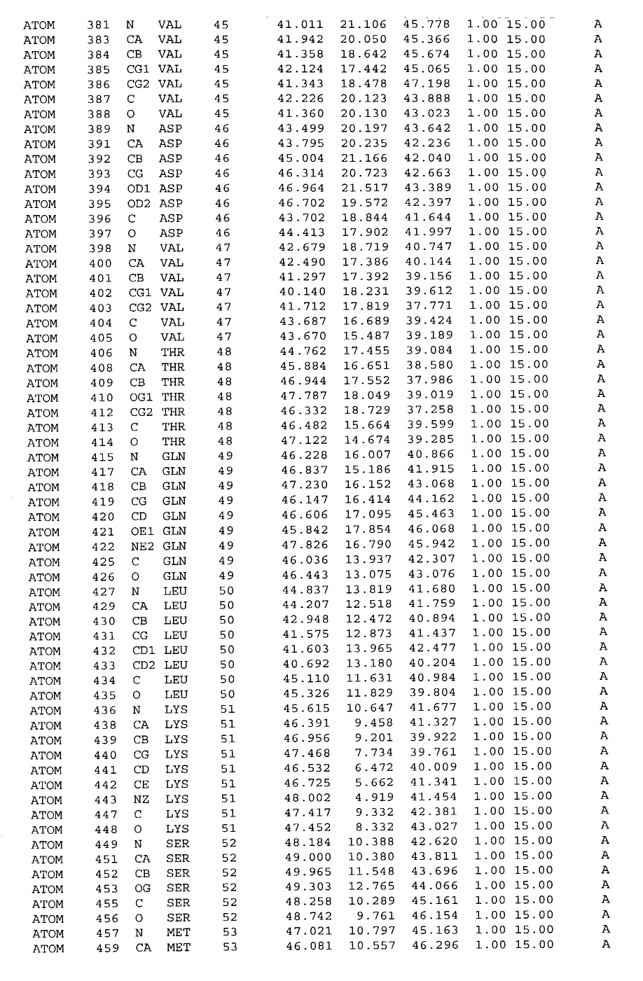

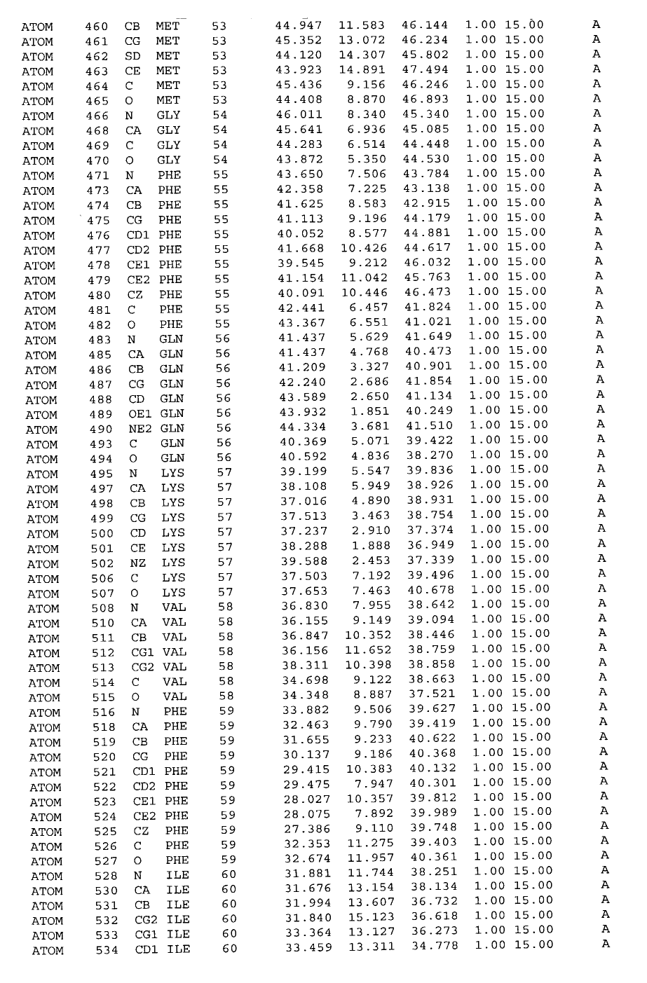

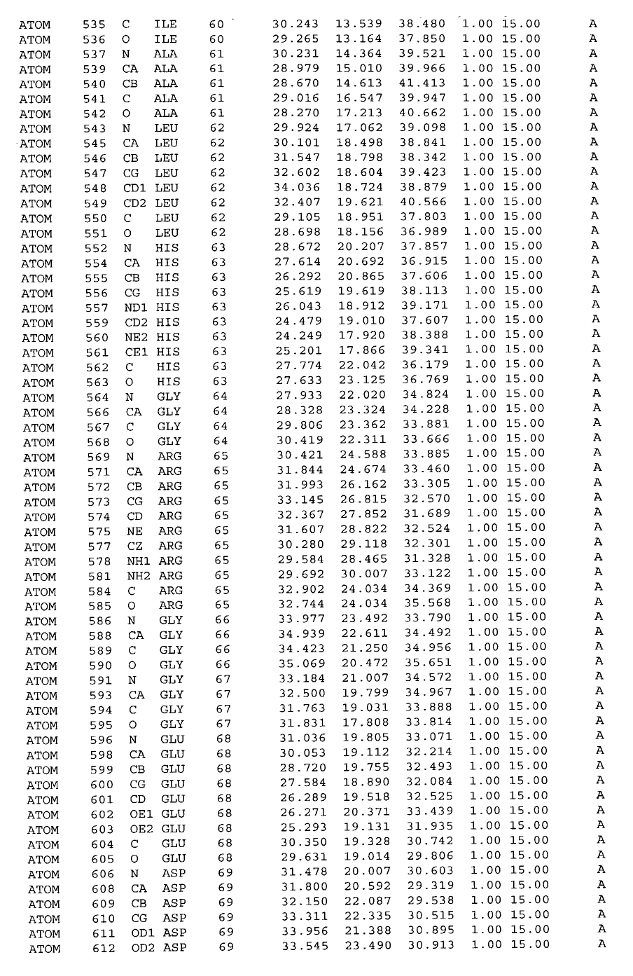

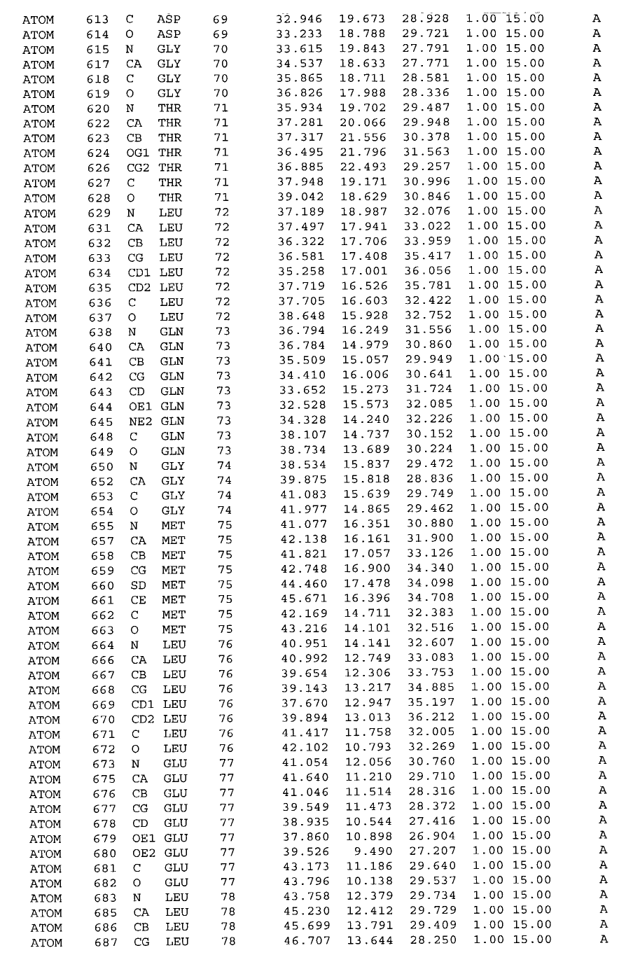

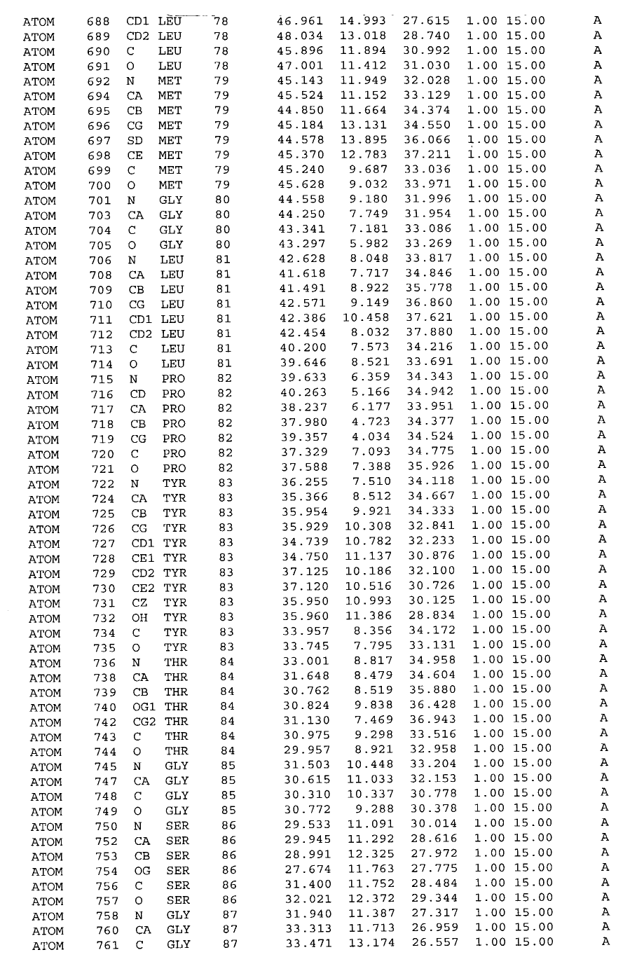

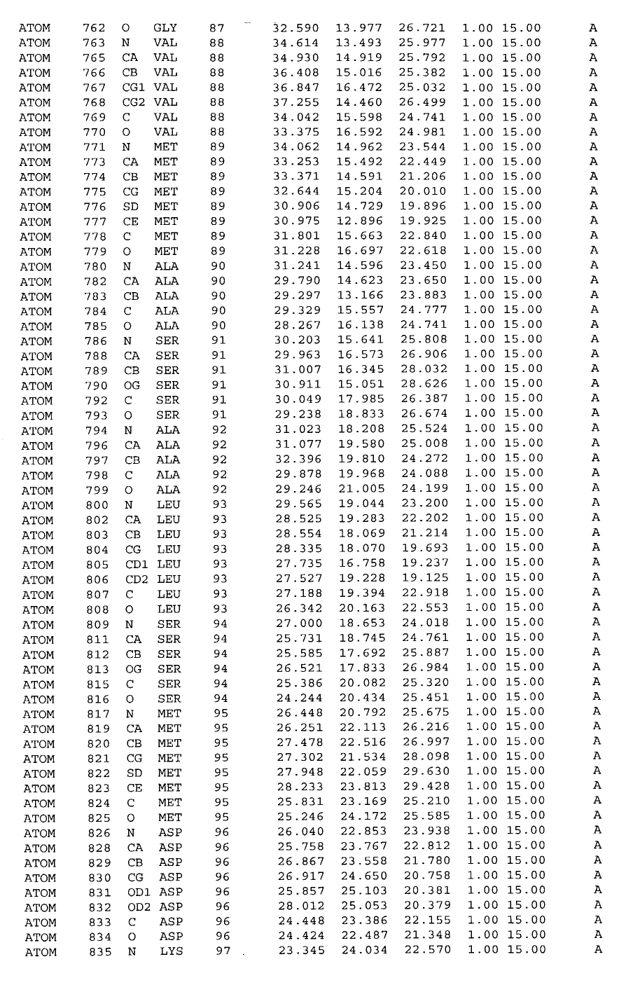

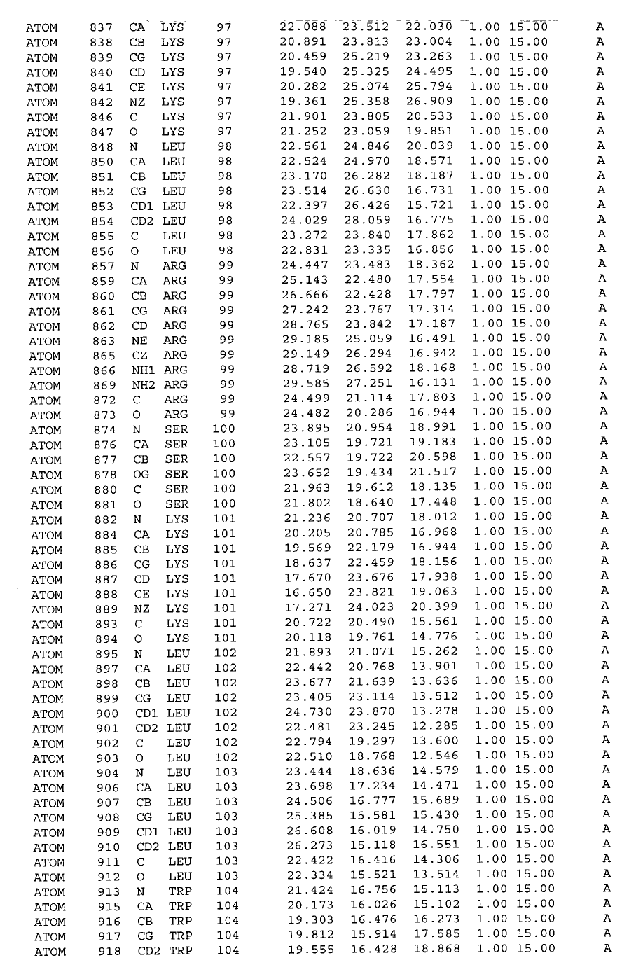

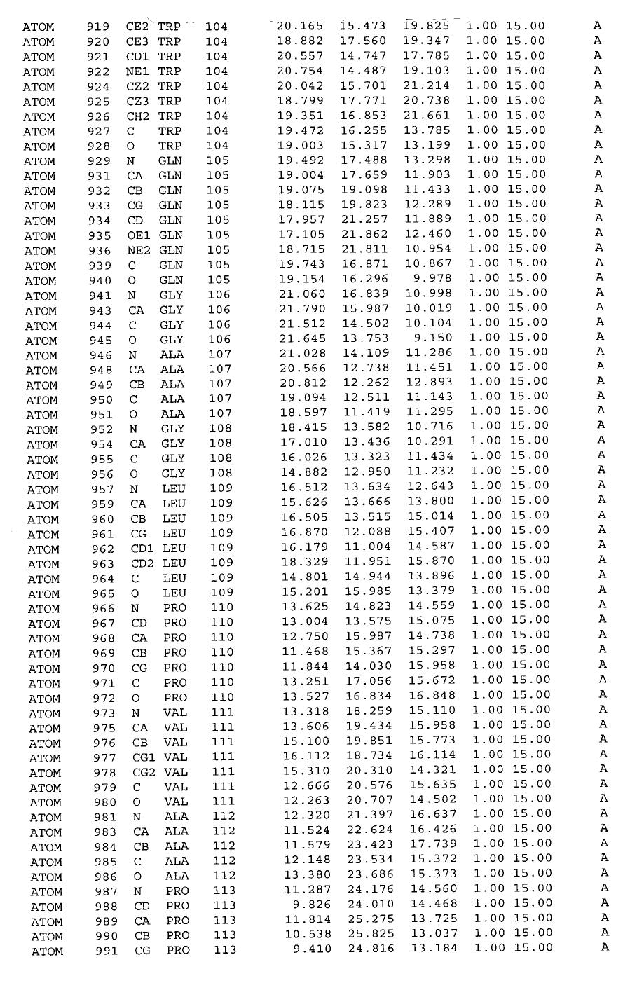

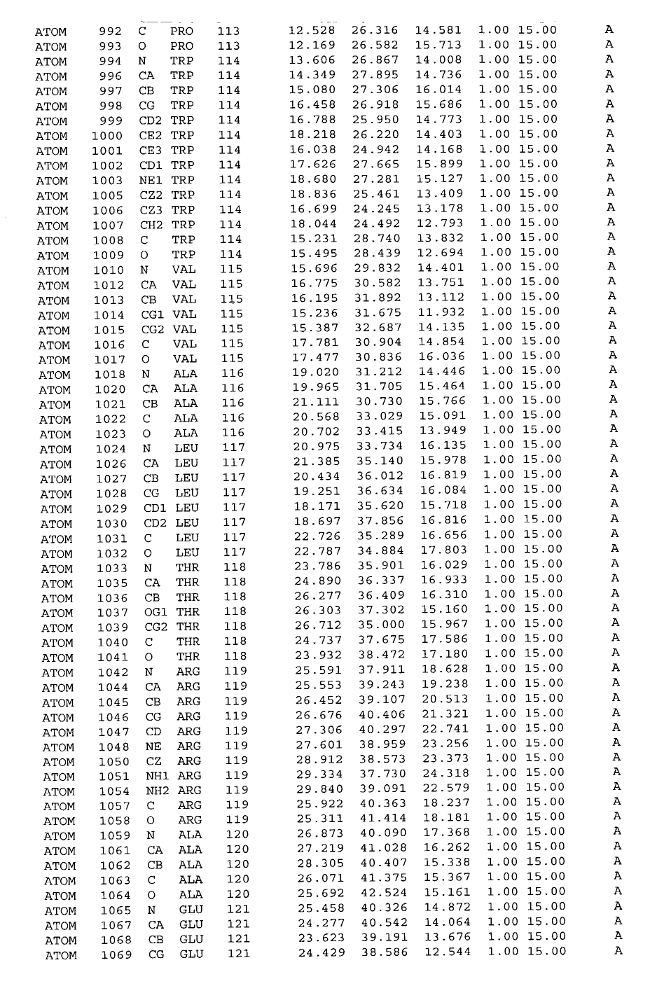

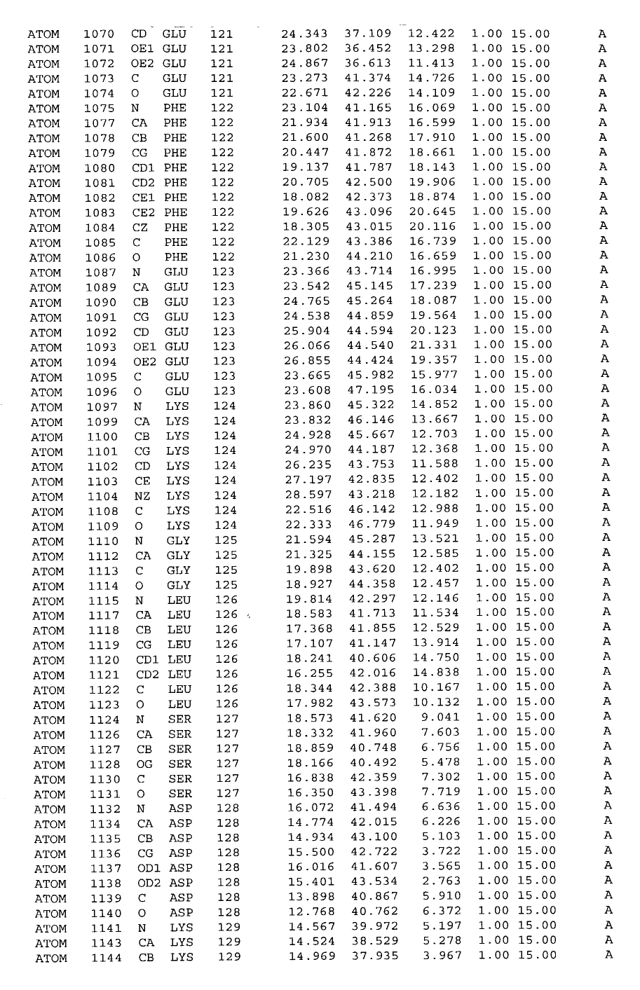

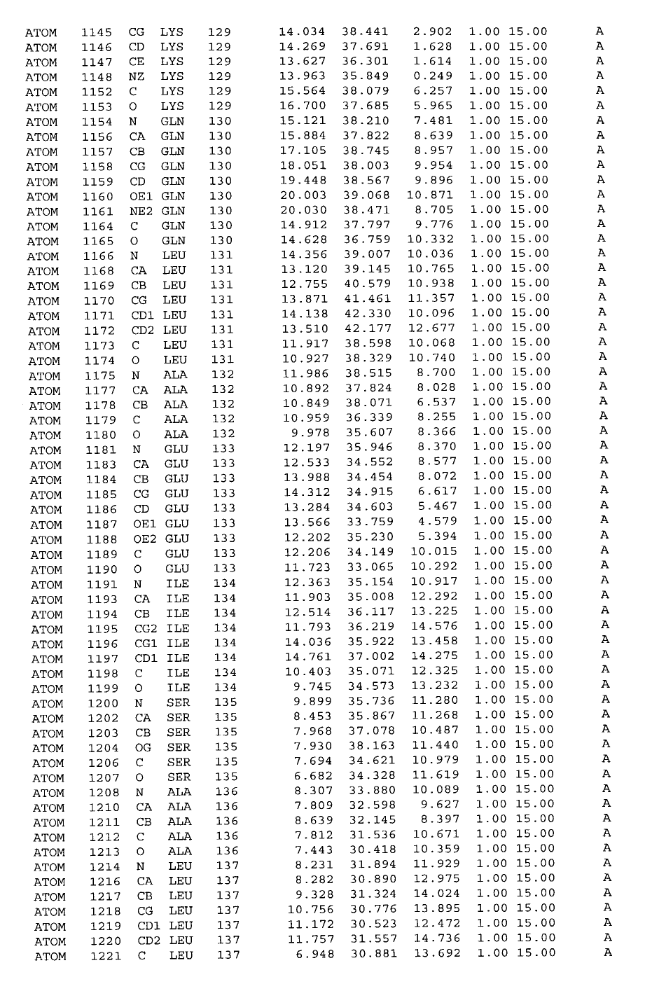

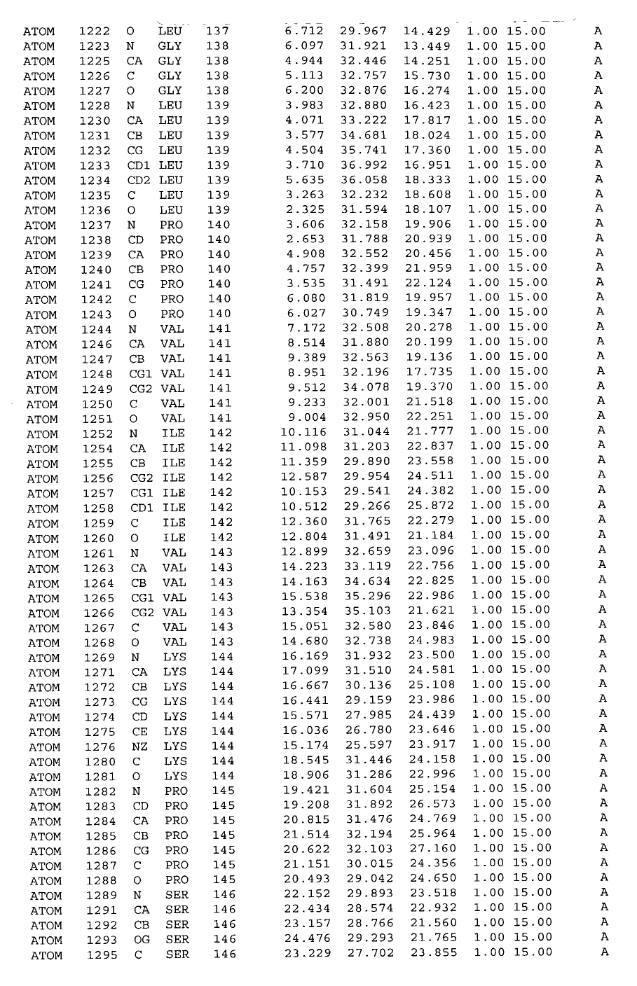

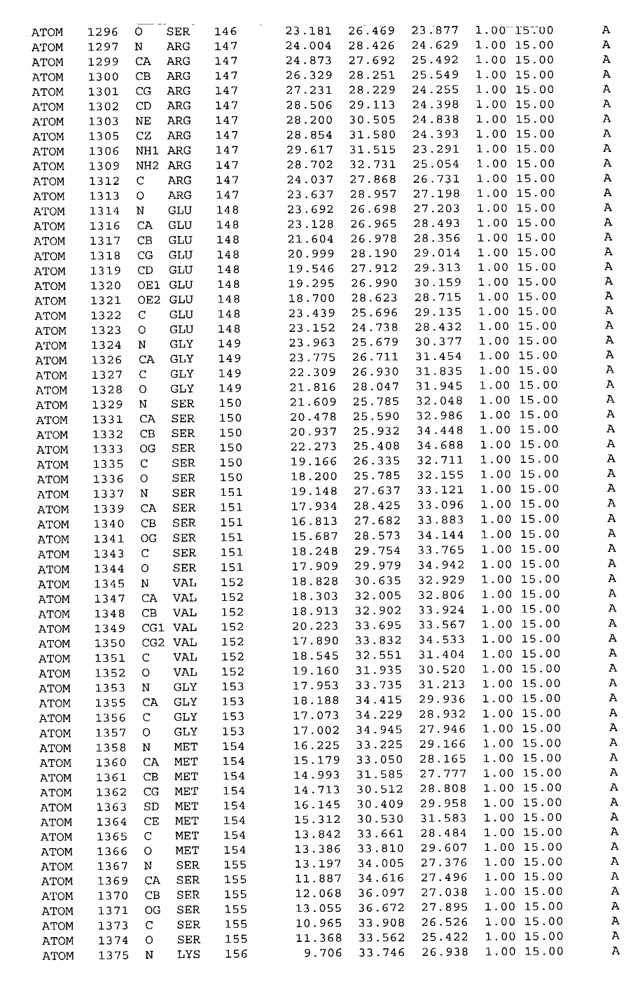

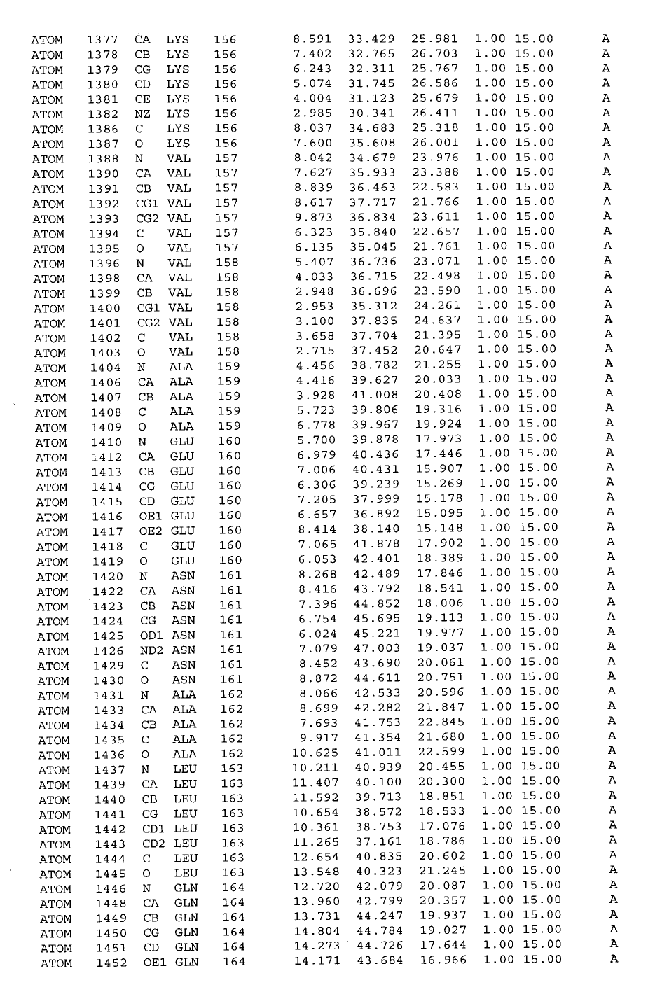

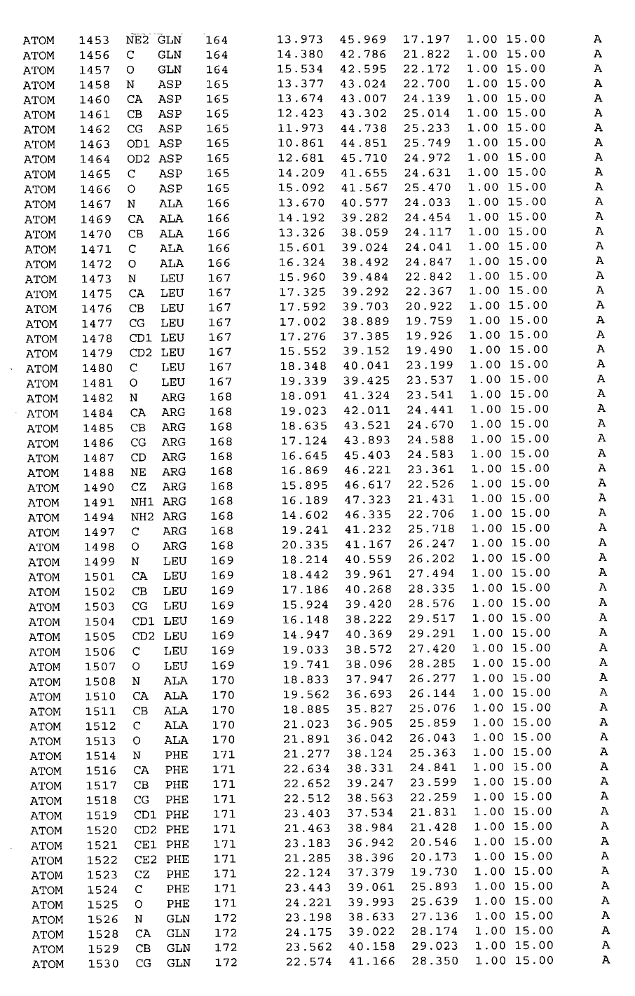

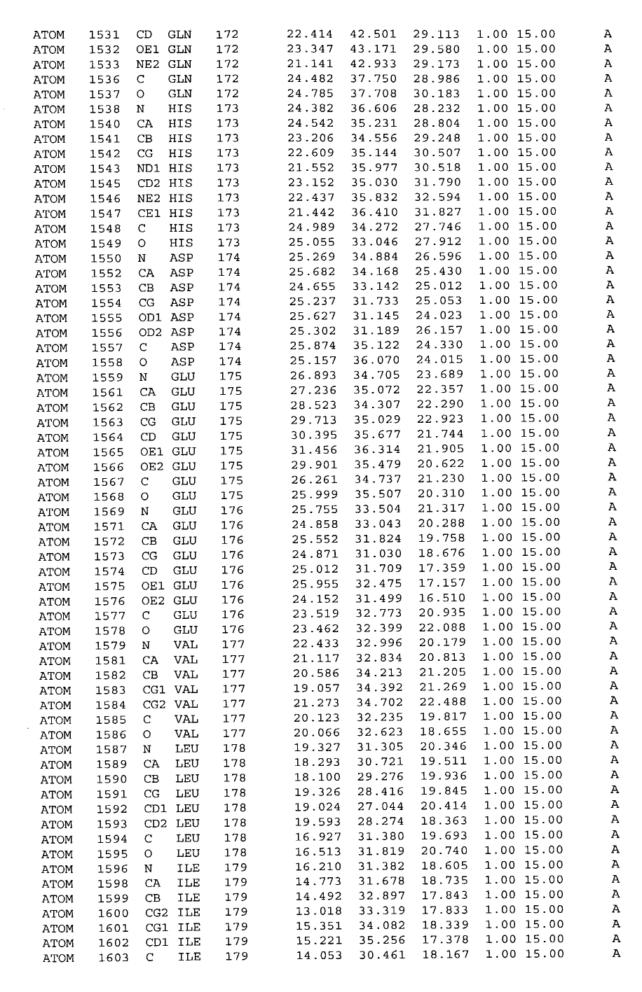

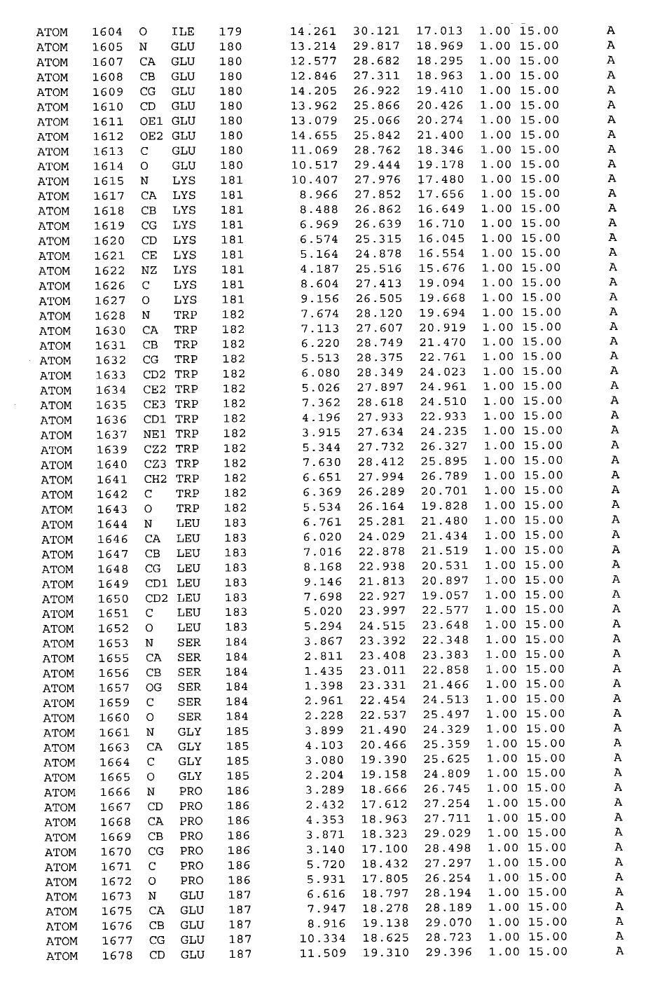

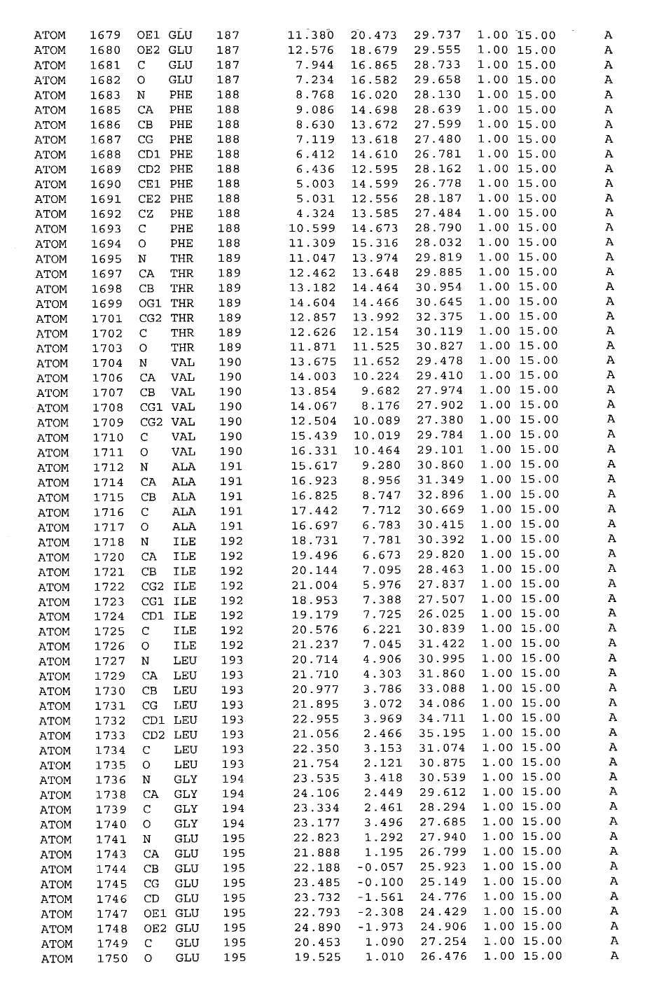

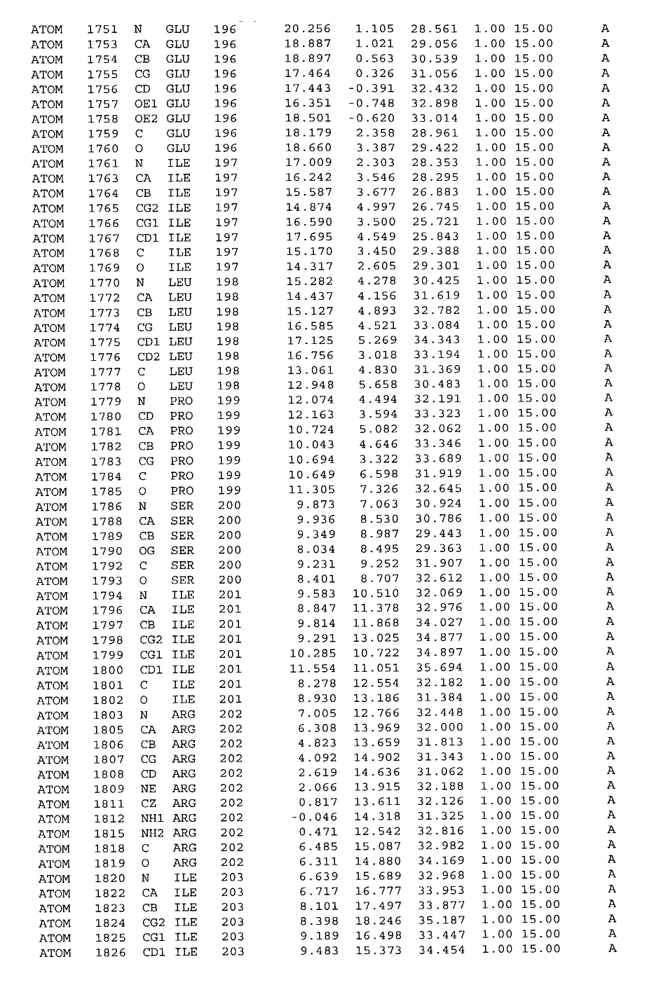

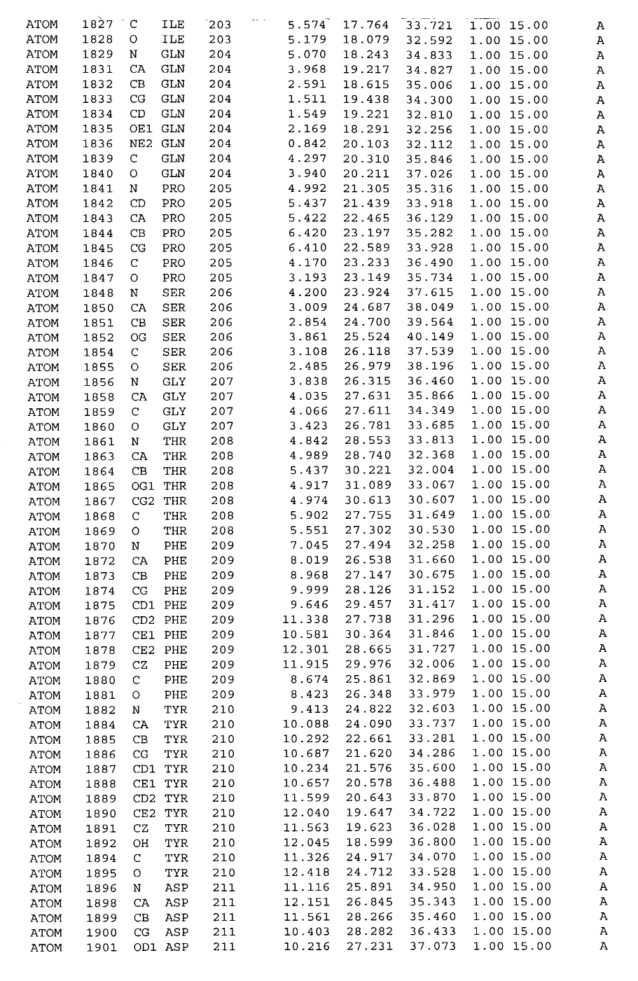

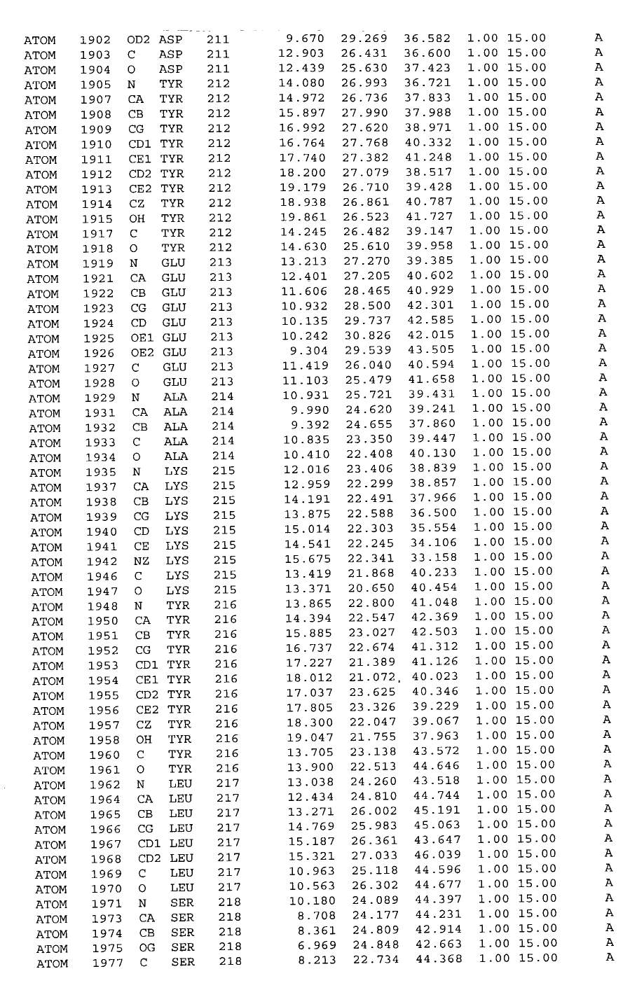

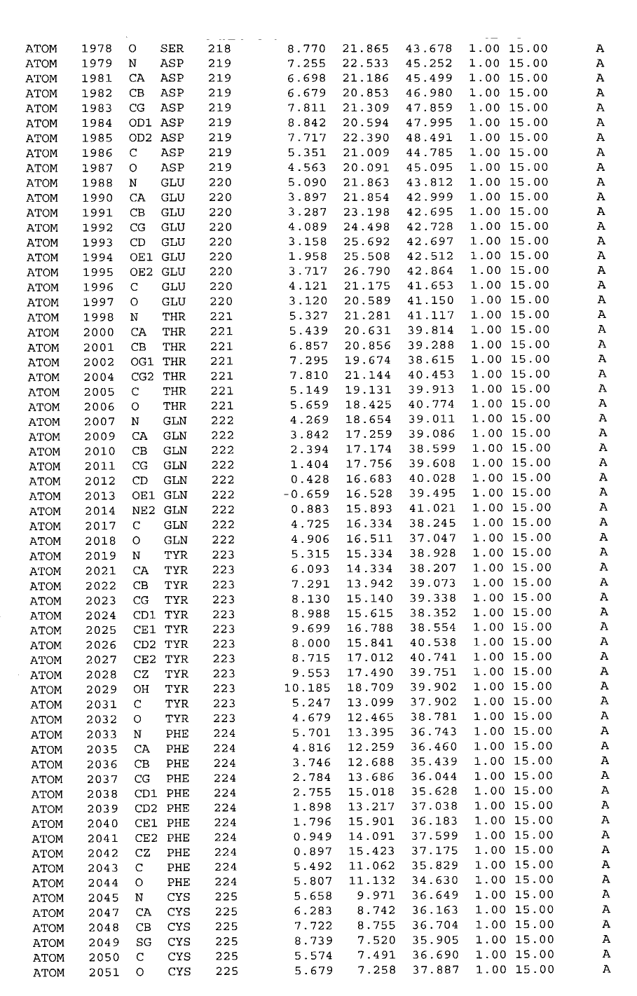

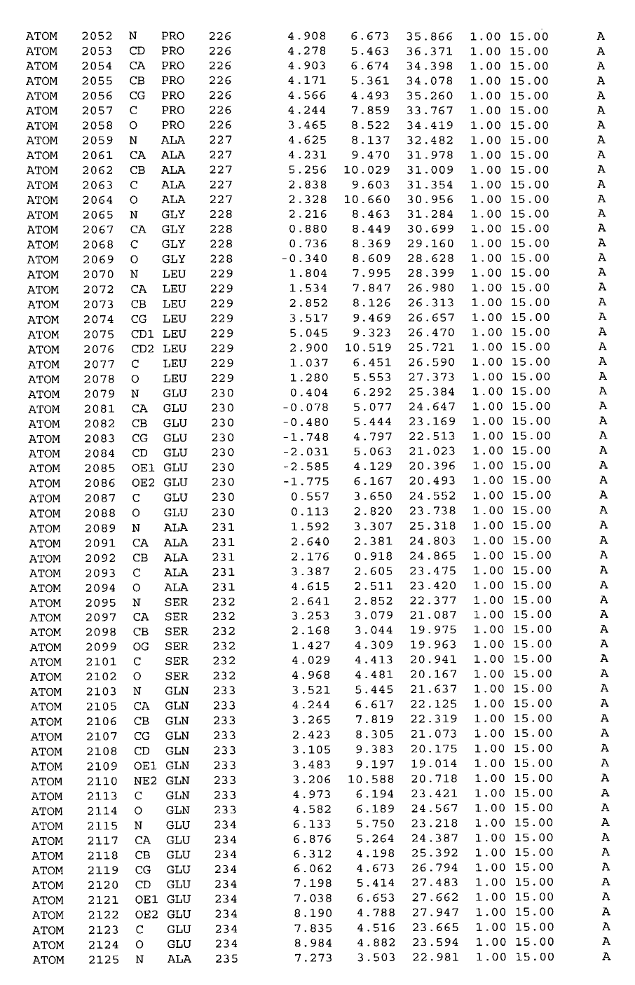

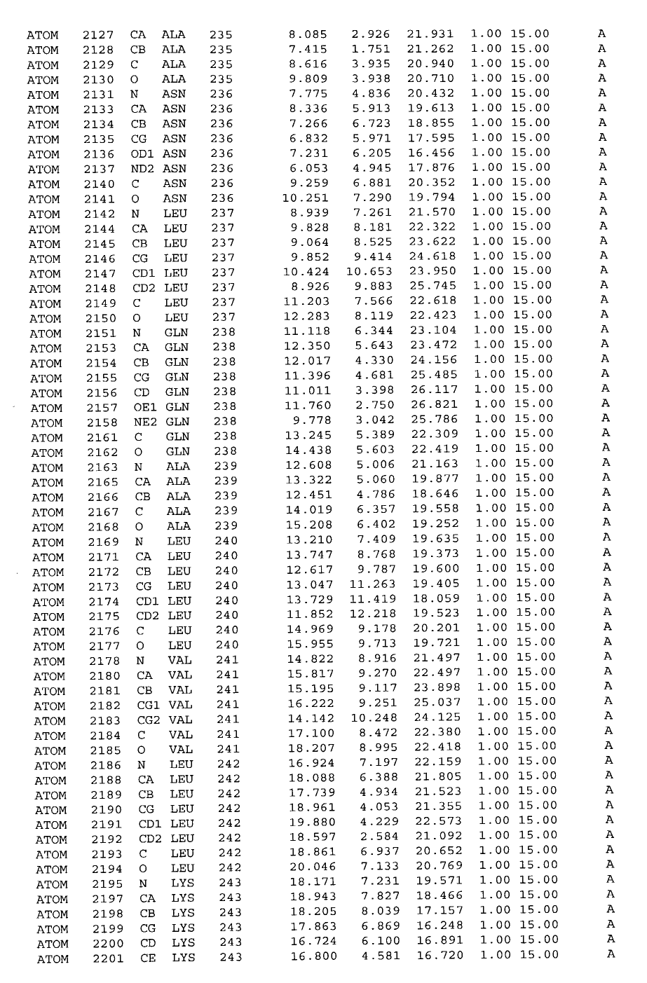

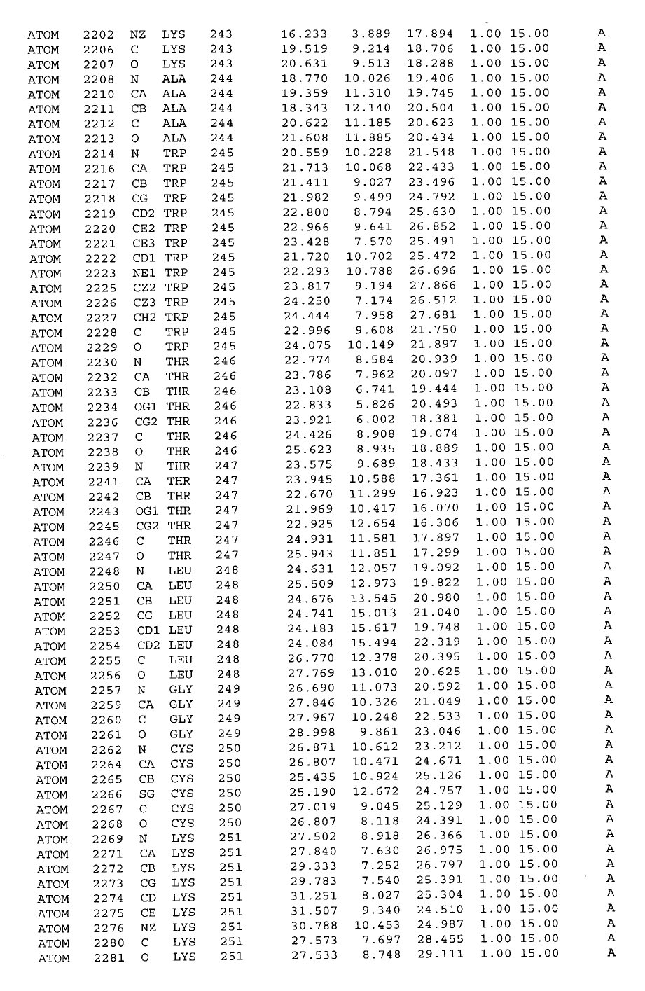

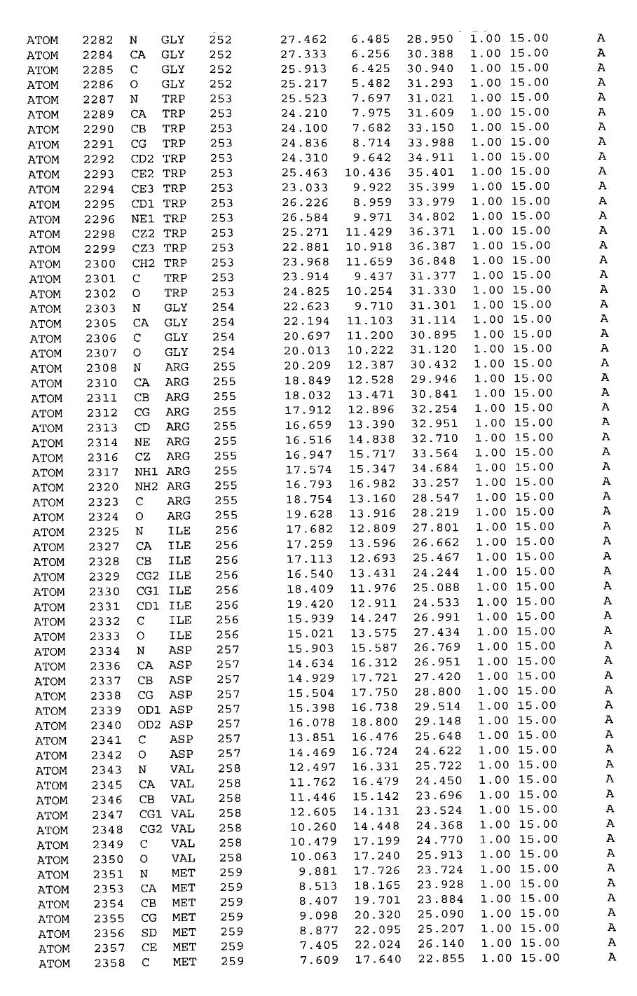

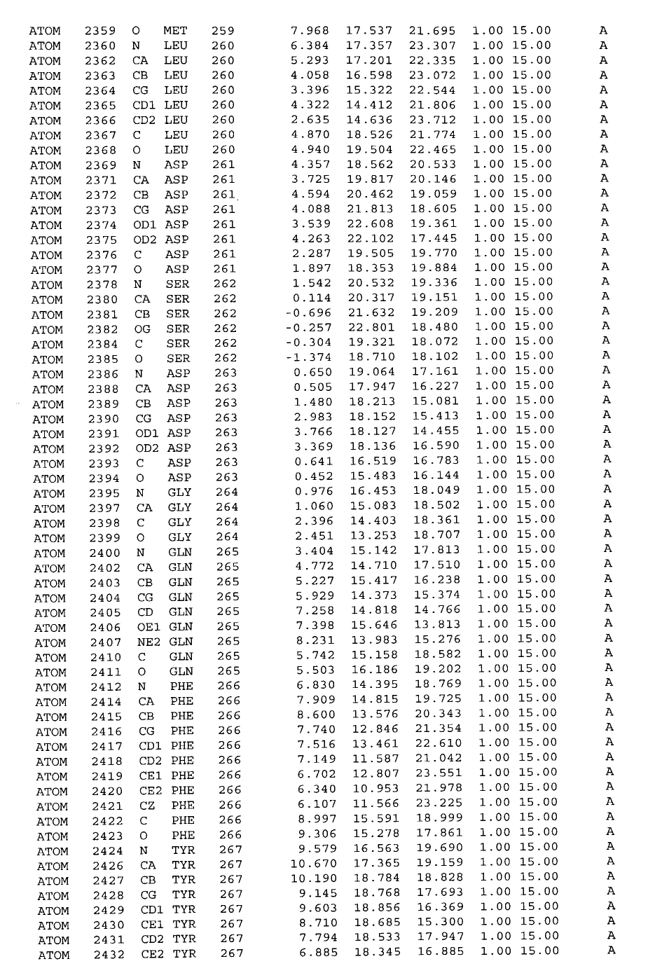

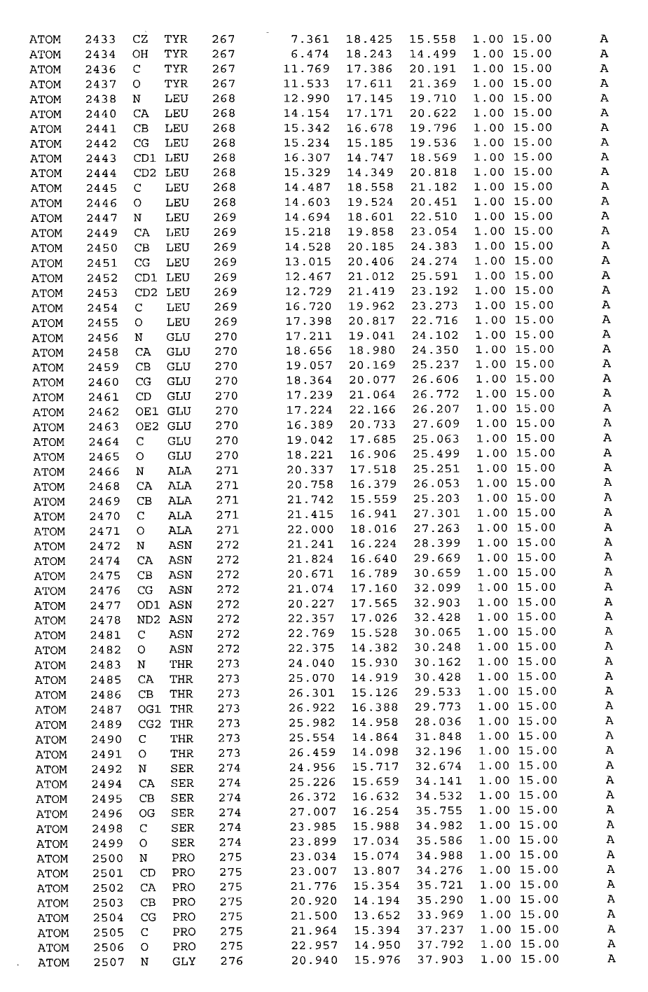

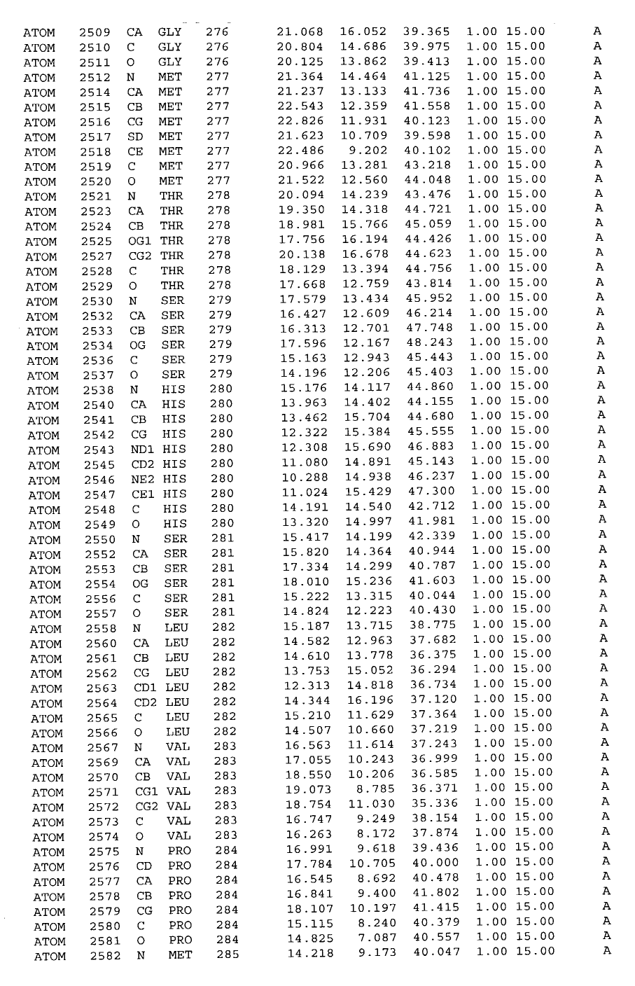

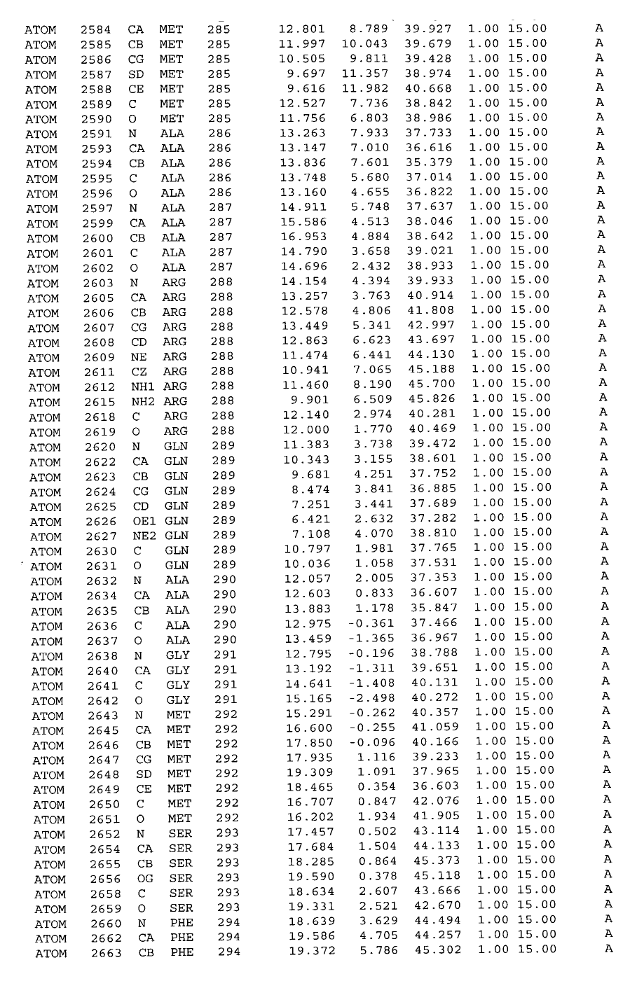

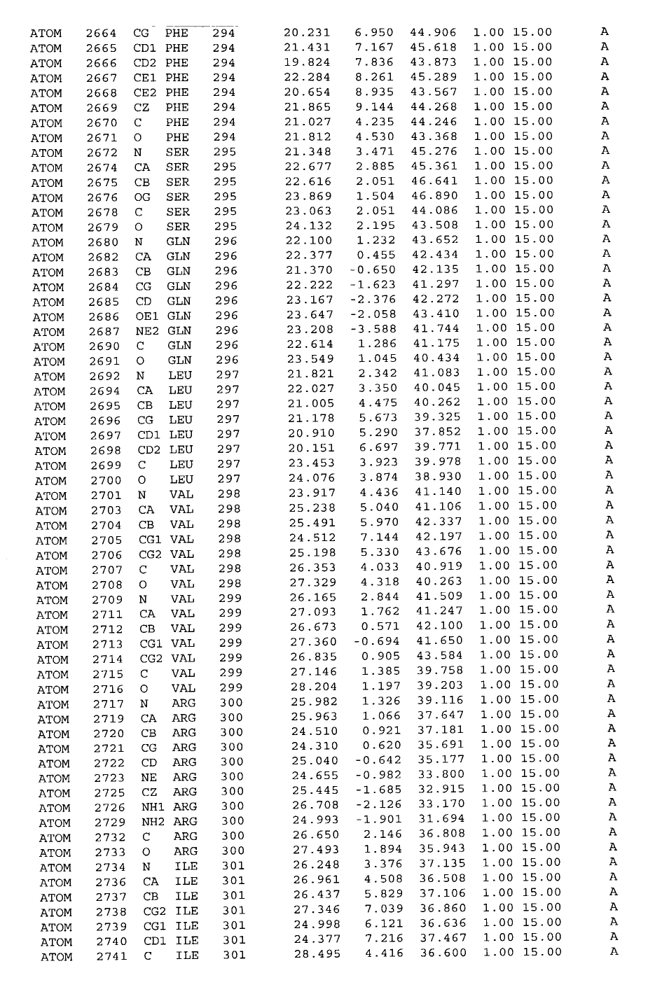

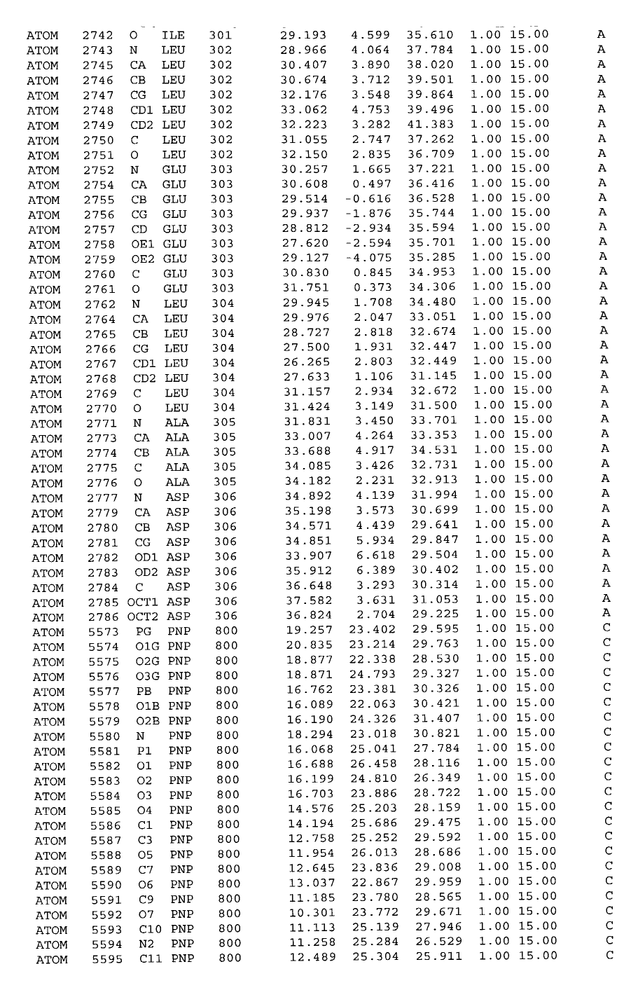

In addition to stopped flow work, steady state fluorescent titration studies can be used to determine the affinity of new compounds for D-Ala-D-Ala ligase. These experiments also utilize the intrinsic tryptophan quenching that occurs upon nucleotide binding. We have determined the affinity of ATP for ligase at 25° C. (FIG. 6). Interestingly, the K_{D} of ATP binding is weaker than the Kₘ, unexpectedly indicating that the rate-limiting step in the ligase mechanism occurs after formation of the products. This methodology can be used to characterize potential inhibitors of ligase. The titration experiments can be carried out, for example, using the methods described in Lohman, T. M. & Mascotti, D. P. (1992) “Nonspecific Ligand-DNA Equilibrium Binding Parameters Determined by Fluorescence Methods” in Methods in Enzymology, vol. 212, p. 425-458.

Proteolysis Experiments

We have developed an in vitro assay to look at the closure of the omega loop (i.e., the D domain). The closure of the omega loop is probed by proteolysis. In the absence of ligands, trypsin cleaves the enzyme into two smaller fragments. The presence of an ATP and phosphinate leads to the protection of this enzyme from proteolysis. This mixture is known to stabilize the closure of the omega loop, as demonstrated by crystallographic studies. ATP or ATP binding molecules alone cannot close the omega loop. However, in the presence of a D-Ala site binding molecule, such as phosphinate, the dipeptide D-Ala-D-Ala, or cycloserine, together with ATP, ADP, or ATPgS stabilize the omega loop closure. Surprisingly, the non-hydrolysable ATP analogue AMPPNP does not support the omega loop closure, possibly indicating a subtle interaction in the phosphate binding region in regard to the closure of the omega loop. We have synthesized an adenosine analogue in which the phosphate group is replaced by a small chain with an amine group at the end. This molecule is of interest for two reasons: it supports the omega loop closure in the presence of phosphinate or cycloserine, and it places in the phosphate binding region a group that enhances the affinity of the molecule. This molecule has a twenty-fold greater affinity over ATP (Kd=300 μM).

Having a molecule that can support the omega loop closure can lead to a significantly higher affinity inhibitor. These studies are also important to determine crystallization conditions at pH 7. At pH 7 only the omega loop closed form of the enzyme appears to crystallize.

Characterization of the conformational change

The crystal structures of the enzyme complexed with our inhibitors clearly reveal a well-defined binding pocket. Certain key interactions between the protein and inhibitor that induce the conformational change are shown in FIG. 4. The residues shown there are key active-site residues that inhibitors have to interact with in order to trigger the large rigid body rotation of domain B towards the active site, as illustrated in FIG. 3. This change can also be described in terms of the movements of individual residues as listed in Table 1.

TABLE 1
The intermolecular distance change during conformational changes:
Distance between residues ILE142 and MET259, and
MET154 and LEU 269 in the hypothetical model EI,
and the crystal structures EI* and EP (closed):

| | ILE142 to MET259 | MET154 and LEU 269 |
|---|---|---|
| EI | 17.4 | 13.5 |
| EI* | 7.9 | 8.9 |
| EP | 7.0 | 8.5 |

Other residues in the active site that we are targeting during the inhibitor optimization process are listed below. These residues can potentially interact directly with inhibitors through van der Waals interactions and/or hydrogen bonds.

Potential hydrophobic interactions with side chains of:

ILE142

TRP182

LEU183

MET259

MET154

LEU269

PHE209

Potential electrostatic interactions with the following side chains (or backbone atoms, where indicated):

GLU180

LYS181

LEU183 (backbone CO)

LEU183 (backbone NH)

GLU185 (backbone NH)

LYS144

GLU187

LYS215

TYR212

SER150

GLU270

ASP257

LYS97

GLU148

ARG255

ASN272

SER94

GLU68

| Residue | Side-chain Interacting Partners |
|---|---|
| Asp | hb donors |
| Glu | hb donors |
| Arg | hb acceptors, aromatic rings |
| Lys | hb acceptors, aromatic rings |
| His | hb donors, hb acceptors, aromatic rings, |
| | positively charged groups |
| Pro | hydrophobic groups (aliphatic, aromatic) |
| Val | hydrophobic groups (aliphatic, aromatic) |
| Ala | hydrophobic groups (aliphatic, aromatic) |
| Leu | hydrophobic groups (aliphatic, aromatic) |
| Ile | hydrophobic groups (aliphatic, aromatic) |
| Trp | hydrophobic groups (aliphatic, aromatic), |
| | positively charged groups |
| Gln | hb donors, hb acceptors |
| Asn | hb donors, hb acceptors |
| Ser | hb donors, hb acceptors |
| Thr | hb donors, hb acceptors |
| Tyr | hb donors, hb acceptors, hydrophobic groups |
| | (aliphatic, aromatic), positively charged groups |
| Phe | hydrophobic groups (aliphatic, aromatic), |
| | positively charged groups |
| Gly | (no side chain) |
| Cys | hb donors, hb acceptors |
| Met | hb donors, hydrophobic groups (aliphatic, aromatic) |

### Processes for Optimizing Inhibitor Potency

We have developed an iterative process for improving the potency of compounds that induce the conformational change described above. The process sequentially utilizes information obtained from protein crystallography, molecular modeling, chemistry, and biochemistry.

Protein Crystallography

The first step in this process is to crystallize and solve the structure of the protein in complex with a ligand that induces the desired conformational change. The binding pocket, in the vicinity of the inhibitor, is analyzed and the structural information can then be used for the design of derivatives tailored to achieve specific interactions with target residues in the catalytic pocket. This approach is best illustrated with the help of a 2D representation of the crystal structure orientation of an inhibitor that we discovered, bound in the active site of D-ala-D-ala ligase, as shown in FIG. 7.

This structure identifies the position 6 of the purine ring as the best anchoring point for effective derivatization, while positions 2, 3, and 9 are involved in crucial interactions with protein residues. Therefore, derivative at position 6 can interact with residues Glu 270 and 187, Asp 157, Lys 144 and 97, and others, as described in the next section.

Molecular Modeling

The structural information of the binding pocket can also be used for the design of optimized analogs by generating and docking virtual libraries of compounds that contain the desired core. For example, based on the crystallography information in FIG. 1, virtual libraries of 6-substituted 2-aminopurines are generated, combining the purine core with commercially available building blocks. The resulting structures are then docked in the active site of D-Ala-D-Ala ligase, and a set of promising compounds is selected on the basis of the docking scores.

As mentioned above, the crystal structure also identifies a series of residues in the binding pocket that could be the potential targets of specific interactions: Glu 270 and 187, Asp 157, Lys 144 and 97 and others. New ligands are designed by derivatizing the purine lead with fragments of the suitable size and chemical features to specifically interact with some of these residues. The design is then validated by docking the resulting derivatives in the catalytic pocket of DDL. The steps involved in the generation and docking of a virtual library of 6-substituted purines are described in example 7.These modeling methods prioritize the synthetic efforts by selecting the most promising candidates for synthesis, thus enhancing the efficiency of the lead optimization process.

Chemistry

The third step in this process is the synthesis of the prioritized compounds. The analogs described above which have been docked into the active site and have prioritized for synthesis base on docking score are then prepared using either proprietary methods or known chemical reactions which have been described in the literature. The virtual compound library described in the Molecular Modeling Section can be created using commercially available starting materials or starting materials described in the literature. In the case in which the starting materials are commercially available, the materials are purchased and then used to synthesize the compounds that have been predicted by docking to be potent enzyme inhibitors. In the case in which the starting materials are not commercially available but have been synthesized as described in the literature, these starting materials are first synthesized using either literature methods or proprietary methods, and then are in turn used to synthesize the chemical structures prioritized by the virtual library docking.

Biochemistry

The final step is to determine if the newly synthesized compounds inhibit the enzyme and then determine if they induce the desired conformational change. Active compounds can be, for example, concurrently tested for activity in an in vitro assay and analyzed by protein crystallography to begin the next round of optimization.

Enzymological studies have been used to deconvolute, or identify, the important components of the ATP binding site. We have discovered that the majority of the affinity comes from the adenine moiety of the ATP molecule and that the phosphates are actually detrimental to the affinity, especially the alpha phosphate. Analysis can, for example, be carried out using the ATPase assay of Duncan et al. (Biochemistry, 27:3709-3714, 1988).

Assays for Inhibition of D-Ala-D-Ala Ligase

Inhibition of D-Ala-D-Ala ligase can be assayed for using the pyruvate kinase/lactate dehydrogenase (PK/LDH) assay described in Example 2. In the bacterial cell wall synthesis process, the ligase catalyzes the conversion of ATP to ADP concurrent with the ligation of two D-alanine residues. PK then regenerates ATP from the ADP thus created simultaneously with the conversion of phosphopyruvate to pyruvate. LDH catalyzes the reduction of pyruvate to lactate by converting NADH to NAD⁺. By monitoring the production rate of NAD⁺, D-Ala-D-Ala ligase activity can be ascertained.

Bisubstrate Analogs

Bisubstrate analogs that not only bind to the ATP-binding site of D-Ala-D-Ala ligase but also bind to the D-Ala binding site are also contemplated. Such analogs would include ATP- and D-Ala-like moieties connected via a flexible or rigid tether (e.g., an alkyl, alkenyl, alkynyl, or polyaromatic connecting group, or a derivative or hybrid of one or more of these groups). Bisubstrate analogs can exhibit increased potency and/or specificity for D-Ala-D-Ala ligase enzymes.

Assays for Antibacterial Activity

The compounds can be screened for antibacterial activity using standard methods.

In one example, illustrated in Example 5, broth microdilution techniques are used to measure in vitro activity of the compounds against a given bacterial culture, to yield minimum inhibitory concentration (MIC) data.

In a typical method, compounds can be screened for antibacterial activity against a plurality of different bacterial strains. Compounds are assayed for potency and breadth of activity in order to identify potential lead compounds. The compounds can be screened for bacteriostatic activity (i.e., prevention of bacterial growth) and/or bactericidal activity (i.e., killing of bacteria).

The lead compounds can be further optimized, for example, by varying substituents to produce derivative compounds. The derivatives can be produced one at a time or can be prepared using parallel or combinatorial synthetic methods. In either case, the derivatives can be assayed to generate structure-activity relationship (SAR) data, which can then be used to further optimize the leads.

Methods for Optimizing for Enzyme Inhibitory Activity

Once a potential inhibitor has been identified (e.g., by comparing the activity of the compound in an enzyme assay to the activity of a standard, such as AMP-PNP), structure-based design methods can be used to optimize the inhibitor. Using drug-like molecules pre-screened in silico with computer models of the active site can enhance the high-throughput screen for lead compounds. For example, the inhibitor and enzyme can be crystallized as a complex and the crystal structure of the complex can be determined. The structural information obtained from the crystal structure can then be used to formulate pharmacophore hypotheses. For example, if the crystal structure indicates, for example, that there is an unexploited hydrogen bond acceptor (e.g., the carbonyl group of a glutamate residue) in the active site of the enzyme a certain distance (e.g., 3 Å) from a hydrogen bond donor (e.g., a protonated amine moiety) of the inhibitor molecule, a new potential inhibitor can be designed, wherein the hydrogen bond donating group is at the appropriate distance. This process can be repeated to provide increasingly potent and specific enzyme inhibitors.

A computational pharmacophore search can be carried out using X-ray crystallographic structural information to generate a computational model. Commercially available compounds can be docked and selected for screening using the docking score as one, but not necessarily the only, element for consideration.

Additional analogs can be bought or synthesized, and then screened. Experiments with these analogs can be used to confirm the hypothesis from the previous screening experiments or to suggest new hypotheses that can similarly be tested by repeating the process. In some cases, alternative templates can be identified and compounds based on these templates can be bought or synthesized to test the new hypotheses. It can be desirable to identify pharmaceutically relevant templates, and/or templates that best test complementary binding hypotheses. In each case, the compounds are typically screened against the enzyme target and also tested for in vitro antibacterial activity.

Moreover, molecular modeling techniques are known in the art, including both hardware and software appropriate for creating and utilizing models of receptors and enzyme conformations.

Numerous computer programs are available and suitable for rational drug design and the processes of computer modeling, model building, and computationally identifying, selecting and evaluating potential antimicrobial compounds in the methods described herein. These include, for example, GRID (available form Oxford University, UK), MCSS (available from Accelrys, Inc., San Diego, Calif.), AUTODOCK (available from Oxford Molecular Group), FLEX X (available from Tripos, St. Louis. Mo.), DOCK (available from University of California, San Francisco), CAVEAT (available from University of California, Berkeley), HOOK (available from Accelrys, Inc., San Diego, Calif.), and 3D database systems such as MACCS-3D (available from MDL Information Systems, San Leandro, Calif.), UNITY (available from Tripos, St. Louis. Mo.), and CATALYST (available from Accelrys, Inc., San Diego, Calif.). Potential antimicrobial compounds may also be computationally designed “de novo” using such software packages as LUDI (available from Biosym Technologies, San Diego, Calif.), LEGEND (available from Accelrys, Inc., San Diego, Calif.), and LEAPFROG (Tripos Associates, St. Louis, Mo.). Compound deformation energy and electrostatic repulsion, may be evaluated using programs such as GAUSSIAN 92, AMBER, QUANTA/CHARMM, AND INSIGHT II/DISCOVER. These computer evaluation and modeling techniques may be performed on any suitable hardware including for example, workstations available from Silicon Graphics, Sun Microsystems, and others. These techniques, methods, hardware and software packages are representative and are not intended to be comprehensive listing. Other modeling techniques known in the art may also be employed in accordance with this invention. See for example, N. C. Cohen, Molecular Modeling in Drug Design, Academic Press (1996) (and references therein), and software identified at various internet sites.

Optimization of D-Ala-D-Ala ligase inhibitory activity can be independent of optimization of antibacterial activity. The different activities can be distinguished by supplying a bacterial strain engineered to overexpress D-Ala-D-Ala ligase (i.e., to create a strain of bacteria that are resistant to D-Ala-D-Ala ligase inhibitors), and then showing that the antibacterial activity of a particular lead compound is not affected by such overexpression.

The invention will be further described in the following examples, which do not limit the scope of the invention described in the claims.

### EXAMPLES

### Example 1

Methods for Protein Crystallization, Data Collection, and Structure Determination

Structural information was obtained by either co-crystallizing D-Ala-D-Ala ligase in the presence of ligands or soaking ligands into pre-formed crystals of the protein. The first approach, produced diffraction quality crystals (hexagonal rods; 0.1 mm×0.1 mm×0.2 mm) of ligase complexed with inhibitors after five days at 18° C. by vapor diffusion in 4 μl drops, containing 5 mg/ml protein, 35 mM acetate buffer (pH 4.5), 2.75% (w/v) polyethylene glycol 6000, 4% DMSO, and a 15-100-fold molar excess of inhibitor over its K₁ value. In the second approach, crystals of ligase in complex with ATP were incubated in a stabilizing solution that contains 70 mM acetate buffer (pH 4.5), 5% (w/v) polyethylene glycol 6000, and a 15-100-fold molar excess of inhibitor over its K₁ value.

Diffraction data was collected at −180° C. on a RAXIS IV ++ imaging plate mounted on a Rigaku RuH3R rotating anode generator equipped with a copper anode, a 0.5 mm cathode, and Osmic mirrors. The unit cell parameters were determined from a single 1° oscillation image, using the DENZO processing software (Z. Otwinowski and W. Minor, “Processing of X-ray Diffraction Data Collected in Oscillation Mode”, Methods in Enzymology, Vol. 276: Macromolecular Crystallography, part A, p. 307-326, 1997, C. W. Carter, Jr. & R. M. Sweet, Eds., Academic Press). Full data sets were obtained from a single crystal by collecting 100-180 oscillation images at 1° intervals for 15 minutes at a detector distance of 100 mm. The co-crystals and soaked-crystals of ligase-inhibitor complexes both belong to the space group P2₁2₁2 with two molecules in the asymmetric unit and the following cell dimensions: a=69.6 Å, b=82.6 Å, and c=96.7 Å. Typical data sets are 98% complete to 2.0 Å with Rsym of 4-9%.

The published atomic coordinates for ligase complexed with the phosphinate inhibitor (Fan et al., Science, 266(5184):439-443, Oct. 21, 1994) were used as a search model to, solve the crystal structure of ligase:AMPPNP by molecular replacement using the XPLOR program (Brunger et al., Science, 235:458-460, 1987), and the refined AMPPNP structure was then used as the starting model to refine subsequent complexes. The structure of ligase complexed with a molecule identified using the methods described herein was refined by performing several cycles of simulated annealing followed by positional and restrained B-factor refinements using XPLOR.

### Example 2

D-Ala-D-Ala-Ligase IC₅₀ Determination

The purine derivatives of Example 1 were dissolved in dimethylsulfoxide (DMSO) at a concentration of 100 mM on the day of screening, using a vortex mixer if necessary for dissolution. The solutions were kept at room temperature until screening was completed.

A 10 mM NADH (Sigma) stock solution was prepared fresh on the day of screening by dissolving 32 μmol NADH in 3.2 ml double-distilled water. The NADH solution was kept on ice. Stock solutions containing 50 mM phosphoenolpyruvate (PEP; Sigma), 500 μM HERMES, 30 mM adenosine triphosphate (ATP; Sigma), 200 mM D-alanine (Sigma), and 4× core buffer (i.e., 100 mM hepes, 40 mM magnesium chloride, and 40 mM potassium chloride), were also prepared and stored on ice. A stock solution of pyruvate kinase/lactate dehydrogenase (PK/LDH) was also obtained from Sigma.

For each set of seven purine test compounds, two 96-well plates were used: an inhibitor plate and an enzyme plate. The test compounds correspond to rows A-G of the plates. D-cycloserine (Sigma), used as a control, corresponds to row H of each plate.

The enzyme solution was allowed to equilibrate to 25° C.

Dilutions were prepared as follows: 50 μl dimethyl sulfoxide (DMSO) was added to each well of columns 1-11, rows A-G, of the inhibitor plate. 50 μl 1×core buffer or DMSO (depending on which solvent the cycloserine control is dissolved in) was added to each well of columns 1-11, row H. 100, of the 100 mM purine solutions were added to column 12, rows A-G (i.e., the first compound in row A, the second compound in row B, and so on). 100 μl of a 100 mM cycloserine solution was added to column 12, row H.

50 μl of solution was transferred from column 12 in each row to column 11 of the same row, mixing the solution with the DMSO. 50 μl of solution was then transferred from column 11 in each row to column 10 in the same row, 50 μl from column 10 was transferred to column 9, and so on, down to column 2. No solution was transferred to column 1. The starting and ending times were noted.

120 μl of the enzyme solution was added to each well of the enzyme plate.

The substrate solutions were brought to 25° C.

The purines and enzymes were then incubated. Since the reactions were initiated in columns, the purines were also added column-by-column to minimize variations in reaction time between wells. At t=0 minutes, 5 μl purine was transferred from each well of columns 1-4 of the inhibitor plate to the corresponding well of the enzyme plate. At t=4 minutes, 5 μl purine was transferred from each well of columns 5-8 of the inhibitor plate to the corresponding well of the enzyme plate. At t=8 minutes, 5 μl purine was transferred from each well of columns 9-12 of the inhibitor plate to the corresponding well of the enzyme plate. The inhibitor plate was then frozen.

At t=18-19 minutes, the substrate solution was taken from 25° C. to a Spectromax® UV-vis spectrophotometer. At t=20 minutes, within a 30 second timeframe, 125 μl of substrate solution was added to each well of columns 1-4, and the absorbance at 340 nm was read. At t=24 minutes and t=28 minutes, respectively, the process was repeated for columns 5-8 and 9-12.

Thus, the concentrations of the compounds in columns 1-12 in each row were 0, 1.9 μM, 3.9 μM, 7.8 μM, 15.6 μM, 31.2 μM, 62.5 μM, 125 μM, 250 μM, 500 μM, 1 mM, and 2 mM, respectively.

The reduction values were multiplied by −4.06 to concert mOD/min units to nM/sec (OD=λLM; λ=6220 1/Mcm; L=0.66 cm; mOD/sec=6220×0.66×(mM/sec)×60; (mOD/sec)×4.06=nM/sec); multiplied by −1 since NADH absorbance decreases as more product is generated).

Plots of reaction rates vs. inhibitor concentration were generated using Kaleidograph®, and IC₅₀ or Kᵢ values were determined after the data was fitted to equations. For % inhibition, enzyme activity in the presence of DMSO was used as a 100% activity reference.

Cycloserine in 1× core buffer has a value of about 150 μM.

This assay method depends on the assumption that the purine compounds are non-competitive inhibitors.

### Example 3

Determination of % Inhibition of D-Ala-D-Ala Ligase

The assay procedure described in Example 2 was repeated, except that inhibitor plates were prepared with 5 mM solutions of the inhibitors in the plates (rather than by serial dilutions), to result in a final concentration of 100 μM inhibitor.

### Example 4

Determination of Kᵢ and Mode of Inhibition

The assay procedure described in Example 2 was repeated, using three different substrate solutions, each in a different enzyme plate. The final concentrations in the reaction mixtures were: (A) 2 mM ATP and 1 mM D-alanine; (B) 2 mM ATP and 32 mM D-alanine; and (C) 50 μM ATP and 32 mM D-alanine. The same inhibitor plate was used with all three enzyme plates. Adenosine (Sigma) and cycloserine (Sigma) were used as controls.

### Example 5

Microdilution Antimicrobial Susceptibility Test Assay

Stock solutions of tested compounds were prepared in DMF at a concentration of 5 mg/ml. Working solutions of the tested compounds were then prepared from the stock solutions, in Mueller-Hinton broth (MHB) with starting concentration of 64 μg/ml (i.e., 25.6 μl of stock solution in 974.4 μl of MHB=128 μg/ml, which was diluted with an equal volume of bacterial inoculum in the procedure that follows).

Bacterial inocula were prepared from overnight culture (i.e., one fresh colony from agar plate in 5 ml MHB; H. influenzae was grown in MHB with the addition of yeast extract, haematin, and NAD), centrifuged 2×5 min/3000 rpm (for S. pneumoniae and H. influenzae, 2×10 min/3000 rpm), and dispensed in 5 ml of fresh MHB each time, such that the bacterial suspension is diluted to obtain 100 colony forming units (cfu) in a microplate well (100 μl total volume).

The microplate wells were then filled with twofold dilutions of tested compound (50 μl), starting with 64 μg/ml. Columns 2-12 were filled with 50 μl of bacterial inoculum (final volume: 100 μl/well). The plates were incubated at 37° C. for 18-24 hours (S. pneumoniae was grown in a C₂-enriched atmosphere).

The optical density of each well at 590 nm (OD₅₉₀) was then measured with a TECAN SpectroFluor Plus®, and minimum inhibitory concentration (MIC) was defined as the concentration that showed 90% inhibition of growth.

### Example 6

MIC determination using overexpressing E. coli

The procedure of Example 5 was repeated, with the following modifications:

The media used for growing bacteria was luria broth (LB) with added antibiotics (20 mg/l chloramphenicol for pBAD vectors, 100 mg/l ampicillin for pTAC vectors for plasmid selection) or M9 minimal media with D-mannitol as a carbon source.

The bacteria used for inoculum in LB were prepared as follows: Overnight culture was diluted 1:50 in a fresh LB media and incubated at 37° C. on a shaker at 250 rpm. After mid-log stage was reached (OD₆₀₀=0.5-1.0, about 3 hours), operon regulator (glucose, arabinose, or IPTG) was added, and the bacteria were further incubated for 3 hours. After 3 hours, OD₆₀₀ was measured again to estimate the bacteria number, and the culture was diluted in LB media (antibiotics—chloramphenicol or ampicillin and regulators were added in double concentrations). Final bacterial inoculum was around 10,000 cfu/well.

The bacteria used for inoculum in M9 minimal media were prepared as follows: Overnight culture in LB was centrifuged 2×5 min/3000 rpm, washed with M9 media, diluted 1:50 in M9 minimal media, left at 37° C. for 14 hours (OD₆₀₀˜0.5), operon regulator was added, and the bacteria were further incubated for 3 hours. After 3 hours, OD₆₀₀ was measured to estimate bacteria number, and the culture was diluted in M9 minimal media (antibiotics—chloramphenicol or ampicillin and regulators were added in double concentrations). The final bacterial inoculum was around 10,000 cfu/well.

Optical density was read out after 24 and 48 hours because of the slower bacterial growth in minimal media.

### Example 7

Docking of a virtual library of 700 purine derivatives

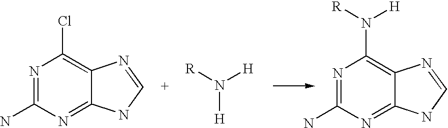

A set of 700 primary aliphatic amines with MW<300, without reactive or toxic functional groups and available from Aldrich is selected from the Available Chemicals Directory (ACD, MDL Information Systems, San Leandro, Calif.).

A library of 700 purines substituted at the 6-position with the selected amines is generated using the Analog Builder module of the Cerius2 program (MSI, Accelrys, Inc., San Diego, Calif.).

A conformational search is performed on the 700 analogs using the Catalyst program (Accelrys, Inc., San Diego, Calif.). A representative set of conformers is thus generated for each compound. Cluster analysis is then performed to reject duplicates. Two conformers of the same molecule are regarded as duplicates if the root mean square deviation between the corresponding coordinates after rigid body superimposition is lower than 1.0 Å. In such cases only one of the two conformers is retained. The selected conformers are docked into the active site of D-Ala-D-Ala ligase with the EUDOC program (provided by Dr. Yuan-Ping Pang, Mayo Clinic). The following Table is representative of the input files used in the docking calculation:

Table of Representative Docking Calculation Input File

| | |
|---|---|
| Search Module (1 = ligand prediction; 2 = virtual screening): | 2 |
| Number of different ligands: | 14258 |
| Box origin on the x-axis: | −44.5 |
| Box origin on the y-axis: | −11.5 |
| Box origin on the z-axis: | 9 |
| Box size on the x-axis: | 9.0 |
| Box size on the y-axis: | 3.5 |
| Box size on the z-axis: | 5.5 |
| Rotational increment (10, 20, or 30 degrees of arc): | 30 |
| Translational increment (0 to 6.0 Å): | 0.5 |
| Cutoff of intermolecular interaction energies (0 to −60 kcal/mol): | 1000.0 |
| Platform (1 = MPP; 2 = Homocluster; 3 = Heterocluster): | 1 |
| Number of available processors: | 10 |

The orientation of each compound with the lowest calculated binding energy is re-scored with a set of 5 additional scoring functions, implemented in the program CSCORE (Tripos, Inc., St. Louis, Mo.), and with the function SCORE (Beijing University). The compounds are ranked based on consensus scoring, and a set of 100 candidates for synthesis is selected accordingly.

### Example 8

D-Ala-D-Ala Ligase Sequence Comparison

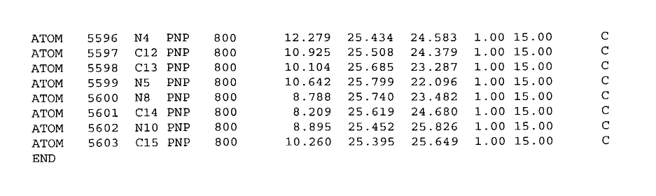

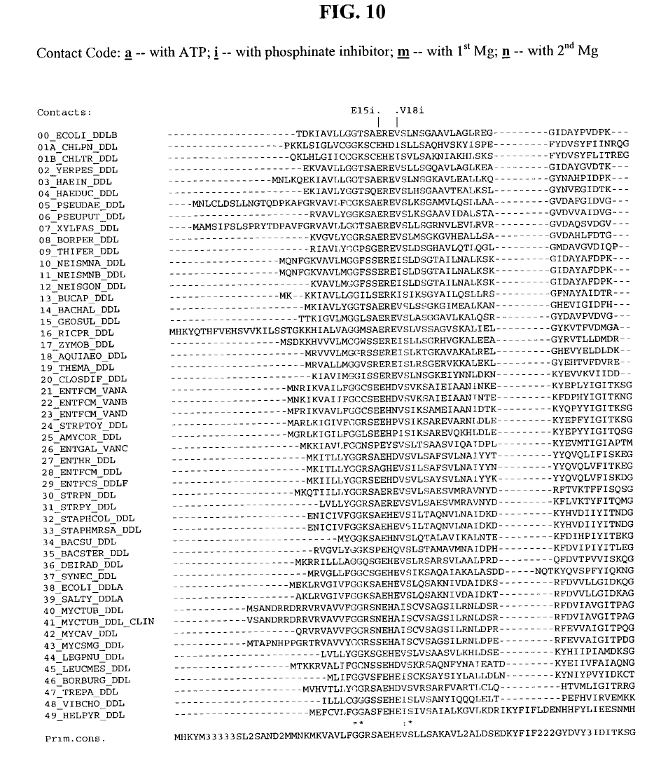

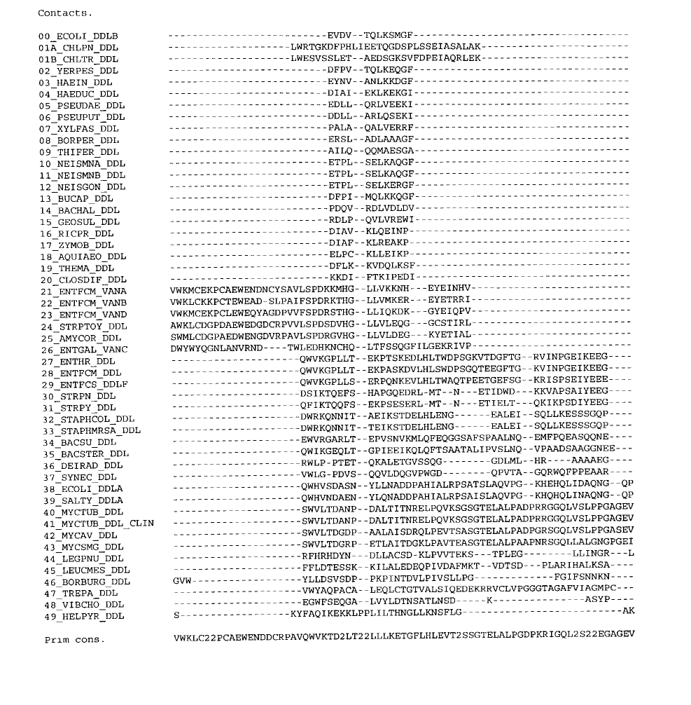

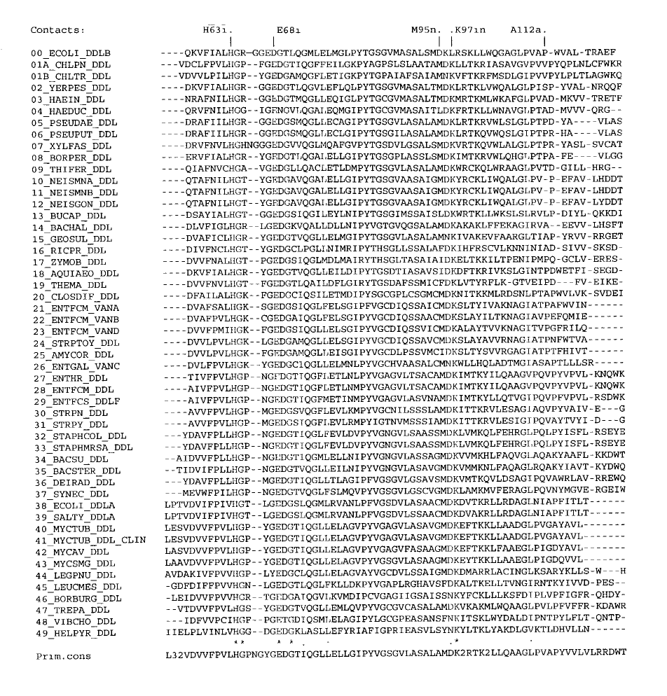

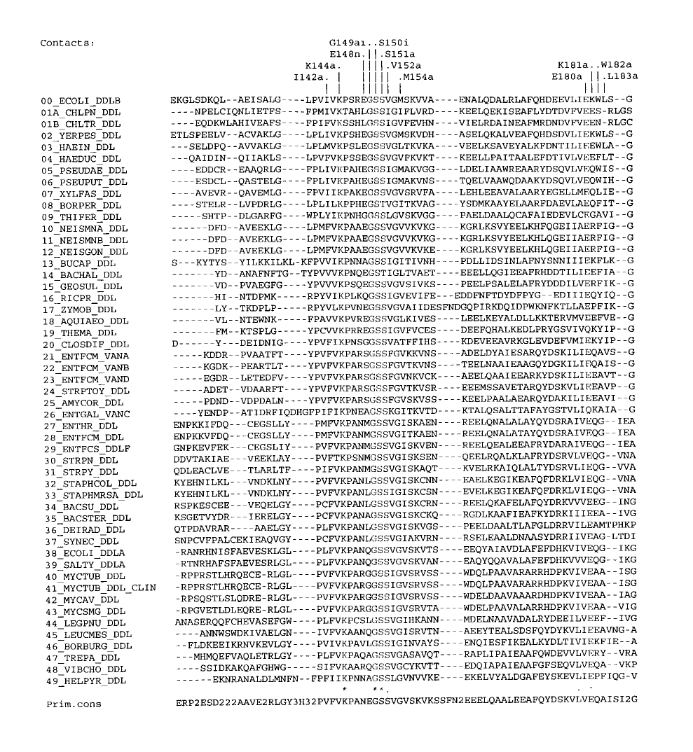

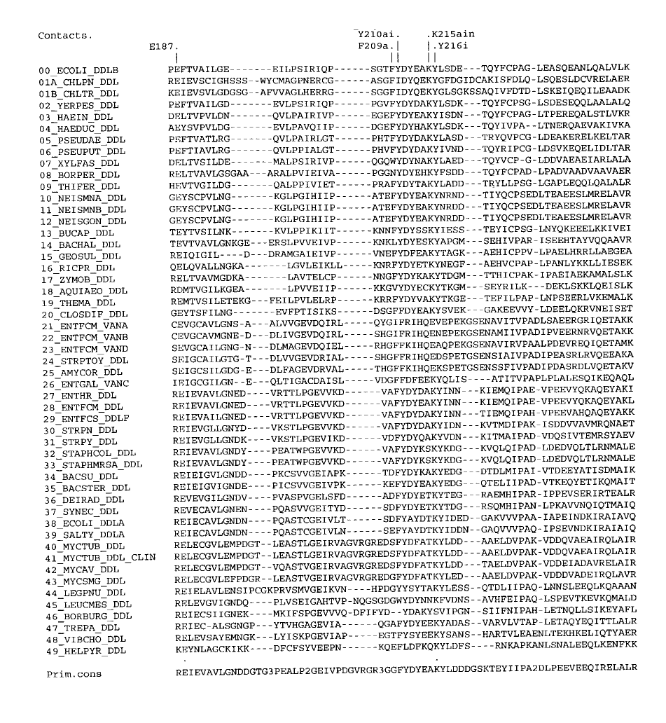

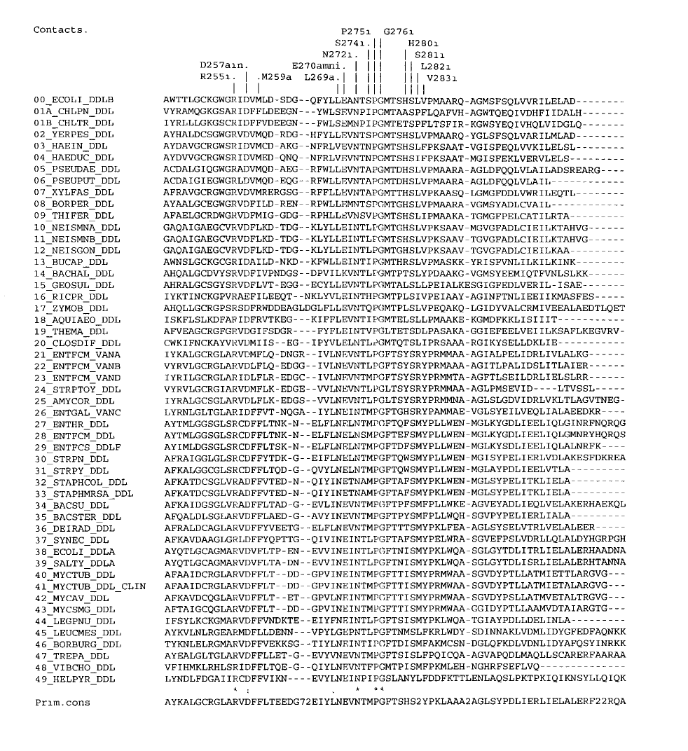

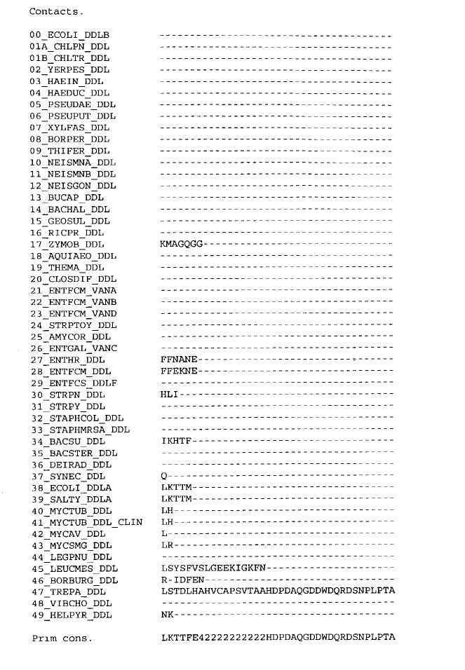

For the following 51 bacterial D-Ala-D-Ala ligase enzymes, we have generated a protein sequence alignment table. The alignment results are shown in FIG. 10. Significant structure elements are indicated in FIG. 10 (see contact codes).

| | |
|---|---|
| Seq 0001: >00_ECOLI_DDLB P07862 | Escherichia coli |
| | (305 res). |
| Seq 0002: >01A_CHLPN_DDL Q9Z701 | Chlamydophila |
| | pneumoniae |
| | (340 res). |
| Seq 0003: >01B_CHLTR_DDL O84767 | Chlamydia |
| | trachomatis |
| | (337 res). |
| Seq 0004: >02_YERPES_DDL Sanger_632 | Yersinia pestis |
| | strain CO-92 chrom |
| | 4 (304 res). |
| Seq 0005: >03_HAEIN_DDL P44405 | Haemophilus |
| | influenzae (306 res). |
| Seq 0006: >04_HAEDUC_DDL HTSC_730 | Haemophilus |
| | ducreyi strain |
| | 35000HP (297 res). |
| Seq 0007: >05_PSEUDAE_DDL 11348402 | Pseudomonas |
| | aeruginosa strain |
| | PAO1 (319 res). |
| Seq 0008: >06_PSEUPUT_DDL TIGR | Pseudomonas putida |
| | KT2440 (292 res). |
| Seq 0009: >07_XYLFAS_DDL 11272188 | Xylella fastidiosa |
| | strain 9a5c |
| | (320 res). |
| Seq 0010: >08_BORPER_DDL Sanger_520 | Bordetella pertussis |
| | Contig845 (296 res). |
| Seq 0011: >09_THIFER_DDL TIGR_6140 | Thiobacillus |
| | ferrooxidans |
| | (296 res). |
| Seq 0012: >10_NEISMNA_DDL 11272192 | Neisseria |
| | meningitidis group |
| | A strain Z2491 |
| | (304 res). |
| Seq 0013: >11_NEISMNB_DDL 11272194 | Neisseria |
| | meningitidis group |
| | B strain MD58 |
| | (304 res). |
| Seq 0014: >12_NEISGON_DDL OUACGT_485 | Neisseria |
| | gonorrhoeae |
| | Ngon_Contig1 |
| | (296 res). |
| Seq 0015: >13_BUCAP_DDL O51927 | Buchnera |
| | aphidicola |
| | (306 res). |
| Seq 0016: >14_BACHAL_DDL 10174238 | Bacillus halodurans |
| | (305 res). |
| Seq 0017: >15_GEOSUL_DDL TIGR_35554 | Geobacter |
| | sulfurreducens |
| | gsulf_5 (299 res). |
| Seq 0018: >16_RICPR_DDL Q9ZDS6 | Rickettsia |
| | prowazekii |
| | (321 res). |
| Seq 0019: >17_ZYMOB_DDL 5834367 | Zymomonas mobilis |
| | (321 res). |
| Seq 0020: >18_AQUIAEO_DDL O66806 | Aquifex aeolicus |
| | thermophile |
| | (291 res). |
| Seq 0021: >19_THEMA_DDL P46805 | Thermotoga |
| | maritima (303 res). |
| Seq 0022: >20_CLOSDIF_DDL Sanger1496 | Clostridium difficile |
| | Contig890 (294 res). |
| Seq 0023: >21_ENTFCM_VANA P25051 | Enterococcus |
| | faecium VanA |
| | (343 res). |
| Seq 0024: >22_ENTFCM_VANB Q06893 | Enterococcus |
| | faecium VanB |
| | (342 res). |
| Seq 0025: >23_ENTFCM_VAND 5353567 | Enterococcus |
| | faecium VanD |
| | (343 res). |
| Seq 0026: >24_STRPTOY_DDL 2228595 | Streptomyces |
| | toyocaensis |
| | (340 res). |
| Seq 0027: >25_AMYCOR_DDL 4405962 | Amycolatopsis |
| | orientalis (348 res). |
| Seq 0028: >26_ENTGAL_VANC P29753 | Enterococcus |
| | gallinarum |
| | (343 res). |
| Seq 0029: >27_ENTHR_DDL Q47827 | Enterococcus hirae |
| | (358 res). |
| Seq 0030: >28_ENTFCM_DDL 12231521 | Enterococcus |
| | faecium |
| | AAG49141.1 |
| | (358 res). |
| Seq 0031: >29_ENTFCS_DDLF Q47758 | Enterococcus |
| | faecalis DDL_f |
| | (348 res). |
| Seq 0032: >30_STRPN_DDL 6634564 | Streptococcus |
| | pneumoniae |
| | (347 res). |
| Seq 0033: >31_STRPY_DDL OUACGT_1315 | Streptococcus |
| | pyogenes Contig_1 |
| | (331 res). |
| Seq 0034: >32_STAPHCOL_DDL TIGR_1280 | Staphylococcus |
| | aureus COL |
| | Contig_8089 |
| | (338 res). |
| Seq 0035: >33_STAPHMRSA_DDL Sanger | Staphylococcus |
| | aureus MRSA |
| | Contig_17 |
| | (338 res). |
| Seq 0036: >34_BACSU_DDL P96612 | Bacillus subtilis |
| | (354 res). |
| Seq 0037: >35_BACSTER_DDL UOKR_1442 | Bacillus |
| | stearothermophilus |
| | Contig_505 |
| | (345 res). |
| Seq 0038: >36_DEIRAD_DDL 7471790 | Deinococcus |
| | radiodurans strain |
| | R1 (339 res). |
| Seq 0039: >37_SYNEC_DDL P73632 | Synechocystis sp. |
| | strain PCC 6803 |
| | (354 res). |
| Seq 0040: >38_ECOLI_DDLA P23844 | Escherichia coli |
| | DDLA (364 res). |
| Seq 0041: >39_SALTY_DDLA P15051 | Salmonella |
| | typhimurium DDLA |
| | (363 res). |
| Seq 0042: >40_MYCTUB_DDL P95114 | Mycobacterium |
| | tuberculosis strain |
| | H37rv (373 res). |
| Seq 0043: >41_MYCTUB_DDL_CLIN TIGR | Mycobacterium |
| | tuberculosis |
| | CSU#93—clinical |
| | (373 res). |
| Seq 0044: >42_MYCAV_DDL TIGR/NIADD | Mycobacterium |
| | avium strain 104 |
| | contig 5490 |
| | (364 res). |
| Seq 0045: >43_MYCSMG_DDL Q9ZGN0 | Mycobacterium |
| | smegmatis (373 res). |
| Seq 0046: >44_LEGPNU_DDL CUCGC_446 | Legionella |
| | pneumophila |
| | (343 res). |
| Seq 0047: >45_LEUCMES_DDL Q48745 | Leuconostoc |
| | mesenteroides |
| | (377 res). |
| Seq 0048: >46_BORBURG_DDL O51218 | Borrelia burgdorferi |
| | strain B31 (356 res). |
| Seq 0049: >47_TREPA_DDL O83676 | Treponema pallidum |
| | (396 res). |
| Seq 0050: >48_VIBCHO_DDL | Vibrio cholerae |
| | strain ASM893 |
| | (319 res). |
| Seq 0051: >49_HELPYR_DDL P56191 | Helicobacter pylori |
| | (347 res). |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for evaluating the potential of a chemical entity to associate with a molecule or molecular complex comprising a binding pocket defined by structural coordinates of D-Ala-D-Ala ligase E. coli amino acids Lys144, Glu180, Lys181, Leu183, Glu187, Asp257, and Glu270 according to FIG. 8; or a homolog of said molecule or molecular complex, wherein said homolog comprises a binding pocket that has a root mean square deviation from the backbone atoms of said amino acids of not more than 10 Å comprising the steps of:
employing computational means to perform a fitting operation between the chemical entity and a binding pocket defined by structural coordinates of D-Ala-D-Ala ligase E. coli amino acids Lys144, Glu180, Lys181, Leu183, Glu187, Asp257, and Glu270 +/− a root mean square deviation from the backbone atoms of said amino acids of not more than 10 Å; and
analyzing the results of said fitting operation to quantify the association between the chemical entity and the binding pocket.

2. A method for identifying a potential inhibitor of D-Ala-D-Ala ligase, the method comprising:
using the atomic coordinates of Lys144, Glu180, Lys 181, Leu183, Glu187, Asp257, and Glu270 of E. coli D-Ala-D-Ala ligase according to FIG. 8 +/− a root mean square deviation from the backbone atoms of said amino acids of not more than 10 Å, to generate a three-dimensional structure of the D-Ala-D-Ala ligase binding pocket;
employing said three-dimensional structure to design or select said potential inhibitor;
synthesizing or obtaining said inhibitor; and
contacting said inhibitor with D-Ala-D-Ala ligase to determine the ability of said potential inhibitor to inhibit D-Ala-D-Ala.

3. The method of claim 2, wherein said employing step comprises designing a molecule that, if docked within said three-dimensional structure, has a hydrogen bond donor between 2.4 and 3.5 Å from one or both carboxylate oxygen atoms of the Glu180 side chain, a hydrogen bond donor between 2.4 and 3.5 Å from the backbone amide oxygen of Lys181, a hydrogen bond acceptor between 2.4 and 3.5 Å from the backbone amide nitrogen of Leu183, a hydrogen bond donor between 2.74 and 3.5 Å from the backbone amide oxygen of Leu183, and a hydrogen bond acceptor between 2.4 and 3.5 Å from the side chain nitrogen of Lys144.

4. The method of claim 3, wherein the molecule further includes hydrophobic interactions 3.5-4.5 Å from the CD1 carbon and SD sulfur atoms of the side chains of Leu269 and Met154, respectively.

5. The method of claim 2, wherein the potential inhibitor is a bisubstrate analog.

6. The method of claim 2, further comprising determining the Ki of the potential inhibitor for the ligase using an enzymatic assay.

7. The method of claim 2, further comprising detecting interactions between the potential inhibitor and the ligase using stopped flow studies.

8. The method of claim 2, further comprising detecting interactions between the potential inhibitor and the ligase by measuring quenching of the ligase's intrinisic tryptophan fluorescence.

9. The method of claim 2, further comprising detecting interactions between the potential inhibitor and the ligase by measuring prevention of proteolysis of the ligase, said prevention being correlated with stabilization of the ligase by the potential inhibitor.

10. The method of claim 2, further comprising determining the effect of the potential inhibitor on bacterial growth of wild-type versus D-Ala-D-Ala ligase-overexpressing strains.

11. A method for identifying a potential inhibitor of D-Ala-D-Ala ligase or a homolog thereof, the method comprising:
designing or selecting a molecule that results in Ile142 of D-Ala-D-Ala ligase or its counterpart in a homolog being brought within 12 Å of Met259 of D-Ala-D-Ala ligase or its counterpart in a homolog, and Met154 of D-Ala-D-Ala ligase or its counterpart in a homolog being brought within 12 Å of Leu269;
synthesizing or obtaining said inhibitor; and
contacting said inhibitor with D-Ala-D-Ala ligase to determine the ability of said potential inhibitor to inhibit D-Ala-D-Ala.

12. The method of claim 11, further comprising determining the Ki of the potential inhibitor for the ligase using an enzymatic assay.

13. The method of claim 11, further comprising detecting interactions between the potential inhibitor and the ligase using stopped flow studies.

14. The method of claim 11, further comprising detecting interactions between the potential inhibitor and the ligase by measuring quenching of the ligase's intrinisic tryptophan fluorescence.

15. The method of claim 11, further comprising detecting interactions between the potential inhibitor and the ligase by measuring prevention of proteolysis of the ligase, said prevention being correlated with stabilization of the ligase by the potential inhibitor.

16. The method of claim 11, further comprising determining the effect of the potential inhibitor on bacterial growth of wild-type versus D-Ala-D-Ala ligase-overexpressing strains.
